SEQUENCE LISTING (7 sequences)

SEQ ID NO: 1 - protein, 10 aa, Homo sapiens
TSAPDTRPAP

SEQ ID NO: 2 - protein, 20 aa, Homo sapiens
AHGVTSAPDTRPAPGSTAPP

SEQ ID NO: 3 - protein, 20 aa, Homo sapiens
AHGVTSAPDNRPALGSTAPP

SEQ ID NO: 4 - protein, 40 aa, Homo sapiens
AHGVTSAPDTRPAPGSTAPPAHGVTSAPDNRPALGSTAPP

SEQ ID NO: 5 - protein, 100 aa, Homo sapiens
AHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPP

SEQ ID NO: 6 - protein, 475 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 7 - protein, 31 aa, Artificial sequence
  TLR2 agonist conjugated MUC-1 peptide
  CARBOHYD (27)..(27): optionally glycosylated with alpha-D-GalNAc
SKKKKGCKLFAVWKITYKDTGTSAPDTRPAP

(19) United States
(12) Patent Application Publication
(10) Pub. No.: US 2021/0220469 A1
(43) Pub. Date: Jul. 22, 2021

(54) Immuno-Oncology Compositions and Methods for Use Thereof

(71) Applicant: GEOVAX, INC., Smyrna, GA (US)

(72) Inventor: Farshad Guirakhoo, Atlanta, GA (US)

(21) Appl. No.: 16/641,728

(22) PCT Filed: Aug. 24, 2018
(86) PCT No.: PCT/US18/47907

§ 371 Date: Feb. 25, 2020

Related U.S. Application Data
(60) Provisional application No. 62/550,374, filed on Aug. 25, 2017.

(51) Int. Cl. A61K 39/285; A61P 35/00; A61K 39/00; C12N 15/86

(52) U.S. Cl. CPC A61K 39/285; A61P 35/00; A61K 2039/5258; C12N 15/86; A61K 2039/575; A61K 39/00117

(57) ABSTRACT

The compositions and methods are described for generating an immune response to a tumor associated antigen such as MUC-1. The compositions and methods described herein relate to a modified vaccinia Ankara (MVA) vector encoding one or more viral antigens for generating a protective immune response to MUC-1 in the subject to which the vector is administered and boosting the immune response by administering a MUC-1 peptide. The compositions and methods of the present invention are useful both prophylactically and therapeutically and may be used to prevent and/or treat neoplasms and associated diseases.

### CROSS REFERENCE TO RELATED APPLICATIONS

This application claims the benefit of U.S. provisional patent application U.S. 62/550,374 filed Aug. 25, 2017, the disclosures of which are hereby incorporated by reference in their entirety.

### FIELD OF THE INVENTION

The compositions and methods described herein relate to compositions, including vaccine compositions, for generating an immune response to hypoglycosylated MUC-1; methods of manufacture; and methods of use thereof. The compositions and methods of the present invention are useful both prophylactically and therapeutically.

### BACKGROUND OF THE INVENTION

In 2016, there will be an estimated 1,735,350 new cancer cases diagnosed and 609,640 cancer deaths in the US (Cancer Facts & FIGURES 2018, American Cancer Society 2018). Cancer vaccines based on human tumor-associated antigens (TAA) have been tested in patients with advanced or recurrent cancer, in combination with or following standard therapy. The immunogenicity and therapeutic efficacy of cancer vaccines has been difficult to properly evaluate due to the multiple highly suppressive effects of the tumor microenvironment and the actions of standard therapy on the patient's immune system. In animal models of human cancer, vaccines administered in the prophylactic setting are most immunogenic and effectively prevent cancer development and progression.

One particular TAA is MUC-1 which is a member of the mucin family and encodes a membrane bound, glycosylated phosphoprotein. MUC-1 has a core protein mass of 120-225 kDa which increases to 250-500 kDa with glycosylation. It extends 200-500 nm beyond the surface of the cell (Brayman M, Thathiah A, Carson D D, 2004, Reprod Biol Endocrinol. 2: 4). The protein is anchored to the apical surface of many epithelia by a transmembrane domain. These repeats are rich in serine, threonine and proline residues which permits heavy o-glycosylation (Brayman M, Thathiah A, Carson DD, 2004, Reprod Biol Endocrinol. 2: 4). Multiple alternatively spliced transcript variants that encode different isoforms of this gene have been reported.

The cytoplasmic tail of MUC-1 is 72 amino acids long and contains several phosphorylation sites (Singh P K, Hollingsworth M A (August 2006), Trends Cell Biol. 16 (9): 467-476). The protein serves a protective function by binding to pathogens and also functions in a cell signaling capacity (Linden S K et al. 2009, PLoS Pathog. 5 (10): e1000617)

Overexpression, aberrant intracellular localization, and changes in glycosylation of this protein have been associated with carcinomas. Specifically, malignant transformation results in loss of polarization and overexpression of aberrantly glycosylated and/or hypoglycosylated MUC-1 which is often associated with colon, breast, ovarian, lung and pancreatic cancers (Gendler S J (July 2001), J. Mammary Gland Biol Neoplasia. 6 (3): 339-353; Scheid E et al. (2016), Cancer Immunol Res 4(10):881-892). Because MUC-1 is abnormally glycosylated in tumor cells, it is subject to immune surveillance resulting in spontaneous induction of anti-tumor antibodies and T cells. The presence of antibodies to altered MUC-1 at diagnosis is associated with clinical benefits [Cramer, D. W., et al., Conditions associated with antibodies against the tumor-associated antigen MUC-1 and their relationship to risk for ovarian cancer. Cancer Epidemiol Biomarkers Prev, 2005. 14(5): p. 1125-31 Since its discovery as a TAA, MUC-1 has been used as a promising antigen for passive (e.g., antibody) and active immunizations (e.g. vaccines) in a number of clinical trials, with some success [Kimura, T. and O. J. Finn, MUC-1 immunotherapy is here to stay. Expert Opin Biol Ther, 2013. 13(1): p. 35-49] Success has been limited by the immunosuppressive microenvironment of advanced cancer that affects cytotoxic and helper T cell responses, upregulation of checkpoint inhibitors (e.g. high expression of PDL1 by tumors and PD1 on responding T cells) and consequently, production of low levels of anti-MUC-1 IgG antibodies. Immunizations against MUC-1 induced some CD8⁺ and CD4⁺ T cell responses in humans and causes tumor regression in preclinical models [Roulois, D., M. Gregoire, and J. F. Fonteneau, MUC-1-specific cytotoxic T lymphocytes in cancer therapy: induction and challenge. Biomed Res Int, 2013. 2013: p. 871936] DNA vaccination with MUC-1 has also shown efficacy in preclinical models [Rong, Y., et al., Induction of protective and therapeutic anti-pancreatic cancer immunity using a reconstructed MUC-1 DNA vaccine. BMC Cancer, 2009. 9: p. 191., Liu, Y. B., et al., [MUC-1-2VNTR DNA Vaccine Induces Immune Responses in Mouse Model with Multiple Myeloma]. Zhongguo Shi Yan Xue Ye Xue Za Zhi, 2015. 23(5): p. 1366-9. Tang, C. K., et al., Oxidized and reduced mannan mediated MUC-1 DNA immunization induce effective anti-tumor responses. Vaccine, 2008. 26(31): p. 3827-34]. Moreover, MVA delivered MUC-1+IL2 (TG4010) was tested in NSCLC, prostate, renal cell carcinoma, and lung cancer, which yielded the best results (6 months improvement vs. chemotherapy but only in Phase 2 studies) [Quoix, E., et al., Therapeutic vaccination with TG4010 and first-line chemotherapy in advanced non-small-cell lung cancer: a controlled phase 2B trial. Lancet Oncol, 2011. 12(12): p. 1125-33]. Currently, MUC-1 antigen is being tested in >60 trials. However, most of these are early phase trials, with only a few in Phase 2b and none in combination with a DNA vaccine, vectored VLP, or ICIs.

Since there is no US approved vaccine for humans against cancer, there is a need for immunogenic vaccine compositions and methods of use to provide the most effective anti-MUC-1 immune responses possible to treat or prevent cancers caused by aberrantly glycosylated or hypoglycosylated MUC-1 expressing tumors.

### SUMMARY OF THE INVENTION

The compositions and methods of the invention described herein are useful for generating an immune response to hypoglycosylated MUC-1 in a subject in need thereof. Advantageously, these compositions and methods may be used prophylactically to immunize a subject against cancer-associated antigens or used therapeutically to treat or ameliorate the onset and severity of disease in a subject in need thereof.

In a first aspect, the present invention is a composition comprising:

a) a recombinant modified vaccinia Ankara (MVA) vector comprising a MUC-1-encoding sequence and a matrix protein-encoding sequence (matrix protein sequence), and

b) a MUC-1 peptide.

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence AHGVTSAPDTRPAPGSTAPPAHGVTSAPDNRPALGSTAPP (SEQ ID NO:4).

In one embodiment, the vectors express an extracellular domain fragment of MUC-1 consisting of the sequence AHGVTSAPDTRPAPGSTAPPAHGVTSAPDTRPAPGSTAPPAHGVTSAPDTRPAPGST APPAHGVTSAPDTRPAPGSTAPPAHGVTSAPDNRPALGSTAPP (Tn-100mer) (SEQ ID NO:5).

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7) wherein the threonine at position 27 is optionally glycosylated with alpha-D-GalNAc.

In one embodiment, the vector comprises a MUC-1 sequence and a matrix protein sequence inserted into one or more deletion sites of the MVA vector.

In one embodiment, the matrix protein is selected from Marburg virus VP40 matrix protein, Ebola virus VP40 matrix protein, human immunodeficiency virus type 1 (HIV-1) matrix protein or Lassa virus matrix Z protein.

In one embodiment, the vector comprises a MUC-1 sequence and a matrix protein sequence inserted into the MVA vector in a natural deletion site, a modified natural deletion site, or between essential or non-essential MVA genes.

In another embodiment, the vector comprises a MUC-1 sequence and a matrix protein sequence inserted into the same natural deletion site, a modified natural deletion site, or between the same essential or non-essential MVA genes.

In another embodiment, the vector comprises a MUC-1 sequence inserted into a deletion site selected from I, II, III, IV, V or VI and a matrix protein sequence is inserted into a deletion site selected from I, II, III, IV, V or VI.

In another embodiment, the vector comprises a MUC-1 sequence and a matrix protein sequence inserted into different natural deletion sites, different modified deletion sites, or between different essential or non-essential MVA genes.

In another embodiment, the vector comprises a MUC-1 sequence inserted in a first deletion site and a matrix protein sequence inserted into a second deletion site.

In a particular embodiment, the vector comprises a MUC-1 sequence inserted between two essential and highly conserved MVA genes; and a matrix protein sequence inserted into a restructured and modified deletion III.

In a particular embodiment, the vector comprises a matrix protein sequence inserted between MVA genes, I8R and G1L.

In a particular embodiment, the vector comprises a MUC-1 sequence inserted between two essential and highly conserved MVA genes to limit the formation of viable deletion mutants.

In a particular embodiment, the vector comprises a MUC-1 sequence inserted between MVA genes, I8R and G1L.

In one embodiment, the promoter is selected from the group consisting of Pm2H5, Psyn II, and mH5 promoters or combinations thereof.

In one embodiment, the recombinant MVA viral vector expresses MUC-1 and matrix proteins that assemble into VLPs.

In a second aspect, the present invention provides a pharmaceutical composition comprising the recombinant MVA vector of the present invention and/or MUC-1 peptide and a pharmaceutically acceptable carrier.

In one embodiment, the recombinant MVA vector is formulated for intraperitoneal, intramuscular, intradermal, epidermal, mucosal or intravenous administration.

In a third aspect, the present invention provides a method of inducing an immune response to a neoplasm in a subject in need thereof, said method comprising:

a) administering a composition comprising an immunogenic vector expressing hypoglycosylated MUC-1 to the subject in an amount sufficient to induce an immune response, or boost a previously induced immune response and

b) administering a composition comprising a MUC-1 peptide in an amount sufficient to induce and immune response or boost a previously induced immune response.

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence.

(SEQ ID NO: 4)
AHGVTSAPDTRPAPGSTAPPAHGVTSAPDNRPALGSTAPP.

In one embodiment, the vectors express an extracellular domain fragment of MUC-1 consisting of the sequence

(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.
(Tn-100 mer)

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7)) wherein the threonine at position 27 is optionally glycosylated with alpha-D-GalNAc.

In one embodiment, the method comprises priming an immune response with an immunogenic vector expressing hypoglycosylated MUC-1 and boosting the immune response with a MUC-1 peptide.

In one embodiment, the immune response is a humoral immune response, a cellular immune response or a combination thereof.

In a particular embodiment, the immune response comprises production of binding antibodies to MUC-1.

In a particular embodiment, the immune response comprises production of neutralizing antibodies to MUC-1.

In a particular embodiment, the immune response comprises production of non-neutralizing antibodies to MUC-1.

In a particular embodiment, the immune response comprises production of a cell-mediated immune response to MUC-1.

In a particular embodiment, the immune response comprises production of neutralizing and non-neutralizing antibodies to MUC-1.

In a particular embodiment, the immune response comprises production of neutralizing antibodies and cell-mediated immunity to MUC-1.

In a particular embodiment, the immune response comprises production of non-neutralizing antibodies and cell-mediated immunity to MUC-1.

In a particular embodiment, the immune response comprises production of neutralizing antibodies, non-neutralizing antibodies, and cell-mediated immunity to MUC-1.

In one embodiment, the neoplasm is selected from leukemia (e.g. myeloblastic, promyelocytic, myelomonocytic, monocytic, erythroleukemia, chronic myelocytic (granulocytic) leukemia, and chronic lymphocytic leukemia), lymphoma (e.g. Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia.

In another embodiment, the neoplasm is selected from Adenocarcinomas (breast, colorectal, pancreatic, other), Carcinoid tumor, Chordoma, Choriocarcinoma, Desmoplastic small round cell tumor (DSRCT), Epithelioid sarcoma, Follicular dendritic cell sarcoma, interdigitating dendritic cell/reticulum cell sarcoma, Lung: type II pneumocyte lesions (type II cell hyperplasia, dysplastic type II cells, apical alveolar hyperplasia), Anaplastic large-cell lymphoma, diffuse large B cell lymphoma (variable), plasmablastic lymphoma, primary effusion lymphoma, Epithelioid mesotheliomas, Myeloma, Plasmacytomas, Perineurioma, Renal cell carcinoma, Synovial sarcoma (epithelial areas), Thymic carcinoma (often), Meningioma or Paget's disease.

In a fourth aspect, the present invention provides a method of treating cancer comprising:

a) administering an effective amount of a recombinant MVA vector expressing hypoglycosylated MUC-1 to prime an immune response, and

b) a MUC-1 peptide in an effective amount to boost an immune response to a subject in need thereof to treat cancer.

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence

(SEQ ID NO: 4)
AHGVTSAPDTRPAPGSTAPPAHGVTSAPDNRPALGSTAPP.

In one embodiment, the vectors express an extracellular domain fragment of MUC-1 consisting of the sequence

(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7)) wherein the threonine at position 27 is optionally glycosylated with alpha-D-GalNAc.

In a fifth aspect, the present invention provides a method of reducing growth of a neoplasm in a subject, said method comprising administering:

a) an effective amount of a recombinant MVA vector expressing hypoglycosylated MUC-1 to prime an immune response, and

b) a MUC-1 peptide in an effective amount to boost an immune response to a subject in need thereof to reduce growth of a neoplasm.

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence

(SEQ ID NO: 4)
AHGVTSAPDTRPAPGSTAPPAHGVTSAPDNRPALGSTAPP.

In one embodiment, the vectors express an extracellular domain fragment of MUC-1 consisting of the sequence

(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7)) wherein the threonine at position 27 is optionally glycosylated with alpha-D-GalNAc.

In a sixth aspect, the present invention provides a method of reducing or preventing growth of a neoplasm in a subject, said method comprising administering:

a) an effective amount of a recombinant MVA vector expressing hypoglycosylated MUC-1 to prime an immune response, and

b) a MUC-1 peptide in an effective amount to boost an immune response to a subject in need thereof to reduce or prevent growth of a neoplasm in the subject.

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence

(SEQ ID NO: 4)
AHGVTSAPDTRPAPGSTAPPAHGVTSAPDNRPALGSTAPP.

In one embodiment, the vectors express an extracellular domain fragment of MUC-1 consisting of the sequence

(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7)) wherein the threonine at position 27 is optionally glycosylated with alpha-D-GalNAc.

In one embodiment, the subject expresses tumor cell markers, but not yet symptomatic. In a particular embodiment, treatment results in prevention of a symptomatic disease.

In another embodiment, the subject expresses tumor cell markers but exhibits minimal symptoms of cancer.

In another embodiment, the method results in amelioration of at least one symptom of cancer.

### BRIEF DESCRIPTION OF THE DRAWINGS

FIG. 1 is an electron micrograph showing virus-like particle (VLP) production by cells infected with MVA-MUC-1VP40, an MVA vaccine encoding MUC-1 TAA protein.

FIG. 2 is a western blot demonstrating that cells infected with the MVA-MUC-1VP40 vaccine (1) express MUC-1 protein, and (2) express hypoglycosylated MUC-1.

FIG. 3 is a graph showing end-point titers of the cross-reactive anti-MUC-1 antibodies assayed with ELISA for sera of non-tumor bearing hMUC-1 transgenic mice immunized with MVA, MTI or a combination of MVA/MTI.

FIG. 4 is a graph showing end-point titers of the cross-reactive anti-MUC-1 antibodies assayed with ELISA for sera of tumor bearing hMUC-1 transgenic mice immunized with MVA, MTI or a combination of MVA/MTI. There is a control group (shown with full circle symbols) from FIG. 3, MVA+MTI, prime/boost from animal of non-tumor bearing hMUC1 transgenic mice.

FIG. 5 is a graph showing tumor size progression after administration of tumor cells in a mouse tumor model with various conditions of MVA, MTI, MVA/MTI and anti-mPD-1.

FIG. 6 is a graph showing end tumor weight measurements in a mouse tumor model with various conditions of MVA, MTI, MVA/MTI and anti-mPD-1.

### DETAILED DESCRIPTION OF THE INVENTION

Compositions and methods are provided to produce an immune response to a hypoglycosylated MUC-1, in a subject in need thereof. The compositions and methods of the present invention can be used to prevent or delay formation of neoplasm or to treat neoplasm or disease associated therewith (such as cancer) in a subject in need thereof. In one embodiment, treatment limits neoplasm development, growth and/or the severity of neoplasm-associated disease such as cancer.

### I. Definitions

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a peptide” includes a plurality of peptides. Thus, for example, a reference to “a method” includes one or more methods, and/or steps of the type described herein, and/or which will become apparent to those persons skilled in the art upon reading this disclosure.

The term “antigen” refers to a substance or molecule, such as a protein, or fragment thereof, that is capable of inducing an immune response.

The term “binding antibody” or “bAb” refers to an antibody which either is purified from, or is present in, a body fluid (e.g., serum or a mucosal secretion) and which recognizes a specific antigen. As used herein, the antibody can be a single antibody or a plurality of antibodies. Binding antibodies comprise neutralizing and non-neutralizing antibodies.

The term “cancer” refers to a malignant neoplasm that has undergone characteristic anaplasia with loss of differentiation, increase rate of growth, invasion of surrounding tissue, and is capable of metastasis.

The term “cell-mediated immune response” refers to the immunological defense provided by lymphocytes, such as the defense provided by sensitized T cell lymphocytes when they directly lyse cells expressing foreign antigens and secrete cytokines (e.g., IFN-gamma.), which can modulate macrophage and natural killer (NK) cell effector functions and augment T cell expansion and differentiation. The cellular immune response is the 2^{nd} branch of the adaptive immune response.

The term “conservative amino acid substitution” refers to substitution of a native amino acid residue with a non-native residue such that there is little or no effect on the size, polarity, charge, hydrophobicity, or hydrophilicity of the amino acid residue at that position, and without resulting in substantially altered immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “deletion” in the context of a polypeptide or protein refers to removal of codons for one or more amino acid residues from the polypeptide or protein sequence, wherein the regions on either side are joined together. The term deletion in the context of a nucleic acid refers to removal of one or more bases from a nucleic acid sequence, wherein the regions on either side are joined together.

The term “Ebola virus” refers to a virus of species Zaire ebolavirus and has the meaning given to it by the International Committee on Taxonomy of Viruses as documented in (Kuhn, J. H. et al. 2010 Arch Virol 155:2083-2103).

The term “fragment” in the context of a proteinaceous agent refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a peptide, polypeptide or protein. In one embodiment the fragment constitutes at least 10%, 20%, 30%, 40%, 50%, 60%, 70%, 80%, or 90% of the entire length of the reference polypeptide. In one embodiment, a fragment of a full-length protein retains activity of the full-length protein. In another embodiment, the fragment of the full-length protein does not retain the activity of the full-length protein.

The term “fragment” in the context of a nucleic acid refers to a nucleic acid comprising an nucleic acid sequence of at least 2 contiguous nucleotides, at least 5 contiguous nucleotides, at least 10 contiguous nucleotides, at least 15 contiguous nucleotides, at least 20 contiguous nucleotides, at least 25 contiguous nucleotides, at least 30 contiguous nucleotides, at least 35 contiguous nucleotides, at least 40 contiguous nucleotides, at least 50 contiguous nucleotides, at least 60 contiguous nucleotides, at least 70 contiguous nucleotides, at least contiguous 80 nucleotides, at least 90 contiguous nucleotides, at least 100 contiguous nucleotides, at least 125 contiguous nucleotides, at least 150 contiguous nucleotides, at least 175 contiguous nucleotides, at least 200 contiguous nucleotides, at least 250 contiguous nucleotides, at least 300 contiguous nucleotides, at least 350 contiguous nucleotides, or at least 380 contiguous nucleotides of the nucleic acid sequence encoding a peptide, polypeptide or protein. In one embodiment the fragment constitutes at least 10%, 20%, 30%, 40%, 50%, 60%, 70%, 80%, or 90% of the entire length of the reference nucleic acid sequence. In a preferred embodiment, a fragment of a nucleic acid encodes a peptide or polypeptide that retains activity of the full-length protein. In another embodiment, the fragment encodes a peptide or polypeptide that of the full-length protein does not retain the activity of the full-length protein.

As used herein, the term “growth inhibitory amount” refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

As used herein, the phrase “heterologous sequence” refers to any nucleic acid, protein, polypeptide or peptide sequence which is not normally associated in nature with another nucleic acid or protein, polypeptide or peptide sequence of interest.

As used herein, the phrase “heterologous gene insert” refers to any nucleic acid sequence that has been or is to be inserted into the recombinant vectors described herein. The heterologous gene insert may refer to only the gene product encoding sequence or may refer to a sequence comprising a promoter, a gene product encoding sequence (such as GP, VP or Z), and any regulatory sequences associated or operably linked therewith.

The term “homopolymer stretch” refers to a sequence comprising at least four of the same nucleotides uninterrupted by any other nucleotide, e.g., GGGG or TTTTTTT.

The term “humoral immune response” refers to the stimulation of Ab production. Humoral immune response also refers to the accessory proteins and events that accompany antibody production, including T helper cell activation and cytokine production, affinity maturation, and memory cell generation. The humoral immune response is one of two branches of the adaptive immune response.

The term “humoral immunity” refers to the immunological defense provided by antibody, such as neutralizing Ab that can directly bind a neoplasm; or, binding Ab that identifies a neoplastic cell for killing by such innate immune responses as complement (C′)-mediated lysis, phagocytosis, and natural killer cells.

The term “immunogenic composition” is a composition that comprises an antigenic molecule where administration of the composition to a subject, results in the development in the subject of a humoral and/or a cellular immune response to the antigenic molecule of interest.

The term “immune response” refers to any response to an antigen or antigenic determinant by the immune system of a subject (e.g., a human). Exemplary immune responses include humoral immune responses (e.g., production of antigen-specific antibodies) and cell-mediated immune responses (e.g., production of antigen-specific T cells). Assays for assessing an immune response are known in the art and may comprise in vivo assays, such as assays to measure antibody responses and delayed type hypersensitivity responses. In an embodiment, the assay to measure antibody responses primarily may measure B-cell function as well as B-cell/T-cell interactions. For the antibody response assay, antibody titers in the blood may be compared following an antigenic challenge. As used herein, “antibody titers” can be defined as the highest dilution in post-immune sera that resulted in a value greater than that of pre-immune samples for each subject. The in vitro assays may comprise determining the ability of cells to divide, or to provide help for other cells to divide, or to release lymphokines and other factors, express markers of activation, and lyse target cells. Lymphocytes in mice and man can be compared in in vitro assays. In an embodiment, the lymphocytes from similar sources such as peripheral blood cells, splenocytes, or lymph node cells, are compared. It is possible, however, to compare lymphocytes from different sources as in the non-limiting example of peripheral blood cells in humans and splenocytes in mice. For the in vitro assay, cells may be purified (e.g., B-cells, T-cells, and macrophages) or left in their natural state (e.g., splenocytes or lymph node cells). Purification may be by any method that gives the desired results. The cells can be tested in vitro for their ability to proliferate using mitogens or specific antigens. The ability of cells to divide in the presence of specific antigens can be determined using a mixed lymphocyte reaction (MLR) assay. Supernatant from the cultured cells can be tested to quantitate the ability of the cells to secrete specific lymphokines. The cells can be removed from culture and tested for their ability to express activation antigens. This can be done by any method that is suitable as in the non-limiting example of using antibodies or ligands which bind to the activation antigen as well as probes that bind the RNA coding for the activation antigen.

The term “improved therapeutic outcome” relative to a subject diagnosed as having a neoplasm or cancer refers to a slowing or diminution in the growth of a tumor, or detectable symptoms associated with tumor growth.

The term “inducing an immune response” means eliciting a humoral response (e.g., the production of antibodies) or a cellular response (e.g., the activation of T cells) directed against hypoglycosylated MUC-1 in a subject to which the composition (e.g., a vaccine) has been administered.

The term “insertion” in the context of a polypeptide or protein refers to the addition of one or more non-native amino acid residues in the polypeptide or protein sequence. Typically, no more than about from 1 to 6 residues (e.g. 1 to 4 residues) are inserted at any one site within the polypeptide or protein molecule.

The term “Marburg virus” refers to a virus of species Marburg marburgvirus and has the meaning given to it by the International Committee on Taxonomy of Viruses as documented in (Kuhn, J. H. et al. 2010 Arch Virol 155:2083-2103).

The term “marker” refers to is meant any protein or polynucleotide having an alteration in expression level or activity that is associated with a disease or disorder.

The term “modified vaccinia Ankara,” “modified vaccinia ankara,” “Modified Vaccinia Ankara,” or “MVA” refers to a highly attenuated strain of vaccinia virus developed by Dr. Anton Mayr by serial passage on chick embryo fibroblast cells; or variants or derivatives thereof. MVA is reviewed in (Mayr, A. et al. 1975 Infection 3:6-14; Swiss Patent No. 568,392).

The term “MTI” as used herein means MUC-1 Tripartite Immunotherapy—a construct having a TLR2 agonist conjugated to a helper epitope conjugated to a MUC-1 epitope for example as described in Lakshminarayanan, V., et al PNAS 109(1):261-266 (2012) and shown diagrammatically below.

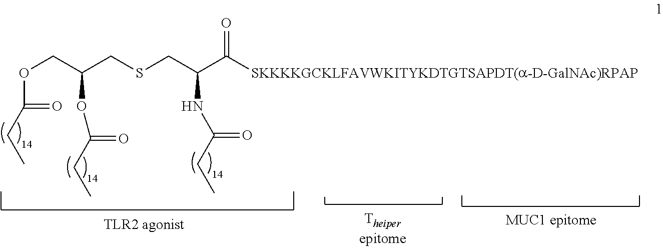

The term “MUC-1 peptide” as used herein means a poly amino acid containing at least 10 consecutive amino acids of the MUC-1 protein sequence. As used herein, MUC-1 peptides include peptide conjugates and hypoglycosylated or non-glycosylated peptides such as for example, but not limited to, MTI and Tn-MUC-1.

The term “neoplasm” as used herein means a new or abnormal growth of tissue in some part of the body especially as a characteristic of cancer.

The term “neutralizing antibody” or “NAb” refers to an antibody which either is purified from, or is present in, a body fluid (e.g., serum or a mucosal secretion) and which recognizes a specific antigen and inhibits the effect(s) of the antigen in the subject (e.g., a human). As used herein, the antibody can be a single antibody or a plurality of antibodies.

The term “non-neutralizing antibody” or “nnAb” refers to a binding antibody that is not a neutralizing antibody.

“Operably linked.” A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “prevent”, “preventing” and “prevention” refers to the inhibition of the development or onset of a condition (e.g., a tumor or a condition associated therewith), or the prevention of the recurrence, onset, or development of one or more symptoms of a condition in a subject resulting from the administration of a therapy or the administration of a combination of therapies.

The term “promoter” refers to a polynucleotide sufficient to direct transcription.

The term “prophylactically effective amount” refers to the amount of a composition (e.g., the recombinant MVA vector or pharmaceutical composition) which is sufficient to result in the prevention of the development, recurrence, or onset of a condition or a symptom thereof (e.g., a tumor or a condition or symptom associated therewith or to enhance or improve the prophylactic effect(s) of another therapy.

The term “recombinant” means a polynucleotide of semisynthetic, or synthetic origin that either does not occur in nature or is linked to another polynucleotide in an arrangement not found in nature.

The term “recombinant,” with respect to a viral vector, means a vector (e.g., a viral genome that has been manipulated in vitro, e.g., using recombinant nucleic acid techniques to express heterologous viral nucleic acid sequences.

The term “regulatory sequence” “regulatory sequences” refers collectively to promoter sequences, polyadenylation signals, transcription termination sequences, upstream regulatory domains, origins of replication, internal ribosome entry sites (“IRES”), enhancers, and the like, which collectively provide for the transcription and translation of a coding sequence. Not all of these control sequences need always be present so long as the selected gene is capable of being transcribed and translated.

The term “shuttle vector” refers to a genetic vector (e.g., a DNA plasmid) that is useful for transferring genetic material from one host system into another. A shuttle vector can replicate alone (without the presence of any other vector) in at least one host (e.g., E. coli). In the context of MVA vector construction, shuttle vectors are usually DNA plasmids that can be manipulated in E. coli and then introduced into cultured cells infected with MVA vectors, resulting in the generation of new recombinant MVA vectors.

The term “silent mutation” means a change in a nucleotide sequence that does not cause a change in the primary structure of the protein encoded by the nucleotide sequence, e.g., a change from AAA (encoding lysine) to AAG (also encoding lysine).

The term “subject” means any mammal, including but not limited to, humans, domestic and farm animals, and zoo, sports, or pet animals, such as dogs, horses, cats, cows, rats, mice, guinea pigs and the like. Determination of those subjects “at risk” can be made by any objective or subjective determination by a diagnostic test or opinion of a subject or health care provider (e.g., genetic test, enzyme or protein marker, marker history, and the like).

The term “surrogate endpoint” means a clinical measurement other than a measurement of clinical benefit that is used as a substitute for a measurement of clinical benefit.

The term “surrogate marker” means a laboratory measurement or physical sign that is used in a clinical or animal trial as a substitute for a clinically meaningful endpoint that is a direct measure of how a subject feels, functions, or survives and is expected to predict the effect of the therapy (Katz, R., NeuroRx 1:189-195 (2004); New drug, antibiotic, and biological drug product regulations; accelerated approval—FDA. Final rule. Fed Regist 57: 58942-58960, 1992.)

The term “surrogate marker for protection” means a surrogate marker that is used in a clinical or animal trial as a substitute for the clinically meaningful endpoint of reduction or prevention of neoplasm growth.

The term “synonymous codon” refers to the use of a codon with a different nucleic acid sequence to encode the same amino acid, e.g., AAA and AAG (both of which encode lysine). Codon optimization changes the codons for a protein to the synonymous codons that are most frequently used by a vector or a host cell.

The term “therapeutically effective amount” means the amount of the composition (e.g., the recombinant MVA vector or pharmaceutical composition) that, when administered to a mammal for treating a neoplasm, is sufficient to effect such treatment for the neoplasm.

The term “treating” or “treat” refer to the eradication or control of a neoplasm, the reduction or amelioration of the progression, severity, and/or duration of a condition or one or more symptoms caused by the neoplasm resulting from the administration of one or more therapies.

The term “vaccine” means material used to provoke an immune response and confer immunity after administration of the material to a subject. Such immunity may include a cellular or humoral immune response that occurs when the subject is exposed to the immunogen after vaccine administration.

The term “vaccine insert” refers to a nucleic acid sequence encoding a heterologous sequence that is operably linked to a promoter for expression when inserted into a recombinant vector. The heterologous sequence may encode a glycoprotein or matrix protein described here.

The term “virus-like particles” or “VLP” refers to a structure which resembles the native virus antigenically and morphologically.

### II. Immunogenic Compositions

Ideal immunogenic compositions or vaccines have the characteristics of safety, efficacy, scope of protection and longevity, however, compositions having fewer than all of these characteristics may still be useful in preventing neoplasm growth or limiting symptoms or disease progression in an exposed subject treated prior to the development of symptoms. In one embodiment the present invention provides a vaccine that permits at least partial, if not complete, protection after a single immunization.

In a first aspect, the present invention is a composition comprising:

a) a recombinant modified vaccinia Ankara (MVA) vector comprising a MUC-1-encoding sequence and a matrix protein-encoding sequence (matrix protein sequence), and

b) a MUC-1 peptide.

In one embodiment, the MUC-1 sequence and the matrix protein sequence are inserted into one or more deletion sites of the MVA vector.

In one embodiment, the composition is a recombinant vaccine or immunogenic vector that comprises one or more nucleic acid sequences encoding hypoglycosylated MUC-1 or immunogenic fragments thereof.

In one embodiment, the composition is a recombinant vaccine or immunogenic vector that comprises an extracellular fragment of MUC-1.

In one embodiment, the composition is a recombinant vaccine or immunogenic vector that comprises an intracellular fragment of MUC-1.

In one embodiment, the composition is a recombinant vaccine or immunogenic vector that comprises an extracellular and an intracellular fragment of MUC-1.

In one embodiment, the composition is a recombinant vaccine or immunogenic vector that comprises an extracellular fragment of MUC-1, an intracellular fragment of MUC-1, and a transmembrane domain of a glycoprotein (GP) of Marburg virus.

In one embodiment, the composition is a recombinant vaccine or immunogenic vector that comprises an extracellular fragment of hypoglycosylated MUC-1, an intracellular fragment of hypoglycosylated MUC-1, and a transmembrane domain of a GP of a virus in the Filoviridae virus family.

In one embodiment, the vector expresses proteins that form VLPs and generate and immune response to hypoglycosylated MUC-1 or immunogenic fragment thereof.

In exemplary embodiments, the immune responses are long-lasting and durable so that repeated boosters are not required, but in one embodiment, one or more administrations of the compositions provided herein are provided to boost the initial primed immune response.

### A. Immunogenic Hypoglycosylated MUC-1 Peptides

The compositions of the present invention are useful for inducing an immune response to hypoglycosylated MUC-1.

In a particular embodiment, the vectors express MUC-1. In one embodiment, the vectors express a glycosylated form of MUC-1. MUC-1 is found on nearly all epithelial cells, but it is over expressed in cancer cells, and its associated glycans are shorter than those of non-tumor-associated MUC-1 (Gaidzik N et al. 2013, Chem Soc Rev. 42 (10): 4421-42).

The transmembrane glycoprotein Mucin 1 (MUC-1) is aberrantly glycosylated and overexpressed in a variety of epithelial cancers and plays a crucial role in progression of the disease. Tumor-associated MUC-1 differs from the MUC-1 expressed in normal cells with regard to its biochemical features, cellular distribution, and function. In cancer cells, MUC-1 participates in intracellular signal transduction pathways and regulates the expression of its target genes at both the transcriptional and post-transcriptional levels (Nath, S., Trends in Mol Med., Volume 20, Issue 6, p 332-342, June 2014)

In one embodiment, the recombinant MVA viral vector expresses MUC-1 and matrix proteins that assemble into VLPs.

In various embodiments, immunogenic fragments of MUC-1 may be expressed by the MVA vectors described herein or administered as peptide or peptide fragments to induce or boost an immune response to MUC-1.

In one embodiment, the MUC-1 peptide is an intracellular domain fragment of MUC-1.

In one embodiment, the MUC-1 peptide is an immunogenic intracellular domain fragment of MUC-1 (for example sequence 407-475 of GenBank Protein Accession Number NP_001191214 or an immunogenic fragment thereof).

In one embodiment, the MUC-1 peptide is an immunogenic extracellular domain fragment of MUC-1 (for example sequence 20-376 of GenBank Protein Accession Number NP_001191214 or an immunogenic fragment thereof).

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence

(SEQ ID NO: 4)
AHGVTSAPDTRPAPGSTAPPAHGVTSAPDNRPALGSTAPP.

In one embodiment, the vectors express an extracellular domain fragment of MUC-1 consisting of the sequence

(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7)) wherein the threonine at position 27 is optionally glycosylated with alpha-D-GalNAc.

### B. Recombinant Viral Vectors Expressing Hypoglycosylated MUC-1 Peptides

Recombinant viral vectors comprising one or more nucleic acid sequences encoding MUC-1 peptides or immunogenic fragments thereof are useful in the methods described herein. In certain embodiments, the recombinant viral vector is a vaccinia viral vector, and more particularly, an MVA vector, comprising one or more nucleic acid sequences encoding hypoglycosylated MUC-1 or immunogenic fragments thereof. Examples of such vectors useful in these methods are described in publication WO2017/120577 incorporated by reference herein.

In a particular embodiment, the vectors express MUC-1. In one embodiment, the vectors express a glycosylated form of MUC-1. MUC-1 is found on nearly all epithelial cells, but it is over expressed in cancer cells, and its associated glycans are shorter than those of non-tumor-associated MUC-1 (Gaidzik N et al. 2013, Chem Soc Rev. 42 (10): 4421-42).

The transmembrane glycoprotein Mucin 1 (MUC-1) is aberrantly glycosylated and overexpressed in a variety of epithelial cancers and plays a crucial role in progression of the disease. Tumor-associated MUC-1 differs from the MUC-1 expressed in normal cells with regard to its biochemical features, cellular distribution, and function. In cancer cells, MUC-1 participates in intracellular signal transduction pathways and regulates the expression of its target genes at both the transcriptional and post-transcriptional levels (Nath, S., Trends in Mol Med., Volume 20, Issue 6, p 332-342, June 2014)

Several such strains of vaccinia virus have been developed to avoid undesired side effects of smallpox vaccination. Thus, a modified vaccinia Ankara (MVA) has been generated by long-term serial passages of the Ankara strain of vaccinia virus (CVA) on chicken embryo fibroblasts (for review see Mayr, A. et al. 1975 Infection 3:6-14; Swiss Patent No. 568,392). The MVA virus is publicly available from American Type Culture Collection as ATCC No.: VR-1508. MVA is distinguished by its great attenuation, as demonstrated by diminished virulence and reduced ability to replicate in primate cells, while maintaining good immunogenicity. The MVA virus has been analyzed to determine alterations in the genome relative to the parental CVA strain. Six major deletions of genomic DNA (deletion I, II, III, IV, V, and VI) totaling 31,000 base pairs have been identified (Meyer, H. et al. 1991 J Gen Virol 72:1031-1038). The resulting MVA virus became severely host cell restricted to avian cells.

Furthermore, MVA is characterized by its extreme attenuation. When tested in a variety of animal models, MVA was proven to be avirulent even in immunosuppressed animals. More importantly, the excellent properties of the MVA strain have been demonstrated in extensive clinical trials (Mayr A. et al. 1978 Zentralbl Bakteriol [B] 167:375-390; Stickl et al. 1974 Dtsch Med Wschr 99:2386-2392). During these studies in over 120,000 humans, including high-risk patients, no side effects were associated with the use of MVA vaccine.

MVA replication in human cells was found to be blocked late in infection preventing the assembly to mature infectious virions. Nevertheless, MVA was able to express viral and recombinant genes at high levels even in non-permissive cells and was proposed to serve as an efficient and exceptionally safe gene expression vector (Sutter, G. and Moss, B. 1992 PNAS USA 89:10847-10851). Additionally, novel vaccinia vector vaccines were established on the basis of MVA having foreign DNA sequences inserted at the site of deletion III within the MVA genome (Sutter, G. et al. 1994 Vaccine 12:1032-1040).

Recombinant MVA vaccinia viruses can be prepared as set out in PCT publication WO2017/120577 incorporated by reference herein. A DNA-construct which contains a DNA-sequence which codes for a foreign polypeptide flanked by MVA DNA sequences adjacent to a predetermined insertion site (e.g. between two conserved essential MVA genes such as I8R/G1L; in restructured and modified deletion III; or at other non-essential sites within the MVA genome) is introduced into cells infected with MVA, to allow homologous recombination. Once the DNA-construct has been introduced into the eukaryotic cell and the foreign DNA has recombined with the viral DNA, it is possible to isolate the desired recombinant vaccinia virus in a manner known per se, preferably with the aid of a marker. The DNA-construct to be inserted can be linear or circular. A plasmid or polymerase chain reaction product is preferred. Such methods of making recombinant MVA vectors are described in PCT publication WO/2006/026667 incorporated by reference herein. The DNA-construct contains sequences flanking the left and the right side of a naturally occurring deletion. The foreign DNA sequence is inserted between the sequences flanking the naturally occurring deletion. For the expression of a DNA sequence or gene, it is necessary for regulatory sequences, which are required for the transcription of the gene, to be present on the DNA. Such regulatory sequences (called promoters) are known to those skilled in the art and include for example those of the vaccinia 11 kDa gene as are described in EP-A-198,328, and those of the 7.5 kDa gene (EP-A-110,385). The DNA-construct can be introduced into the MVA infected cells by transfection, for example by means of calcium phosphate precipitation (Graham et al. 1973 Virol 52:456-467; Wigler et al. 1979 Cell 16:777-785), by means of electroporation (Neumann et al. 1982 EMBO J. 1:841-845), by microinjection (Graessmann et al. 1983 Meth Enzymol 101:482-492), by means of liposomes (Straubinger et al. 1983 Meth Enzymol 101:512-527), by means of spheroplasts (Schaffher 1980 PNAS USA 77:2163-2167) or by other methods known to those skilled in the art.

The MVA vectors described in WO2017/120577 are immunogenic after a single prime or a homologous prime/boost regimen. Other MVA vector designs require a heterologous prime/boost regimen while still other published studies have been unable to induce effective immune responses with MVA vectors. Conversely, these MVA vector are useful in eliciting effective T-cell and antibody immune responses. Furthermore, the utility of an MVA vaccine vector capable of eliciting effective immune responses and antibody production after a single homologous prime boost is significant for considerations such as use, commercialization and transport of materials especially to affected third world locations.

In one embodiment, the present invention is a recombinant viral vector (e.g., an MVA vector) comprising one or more nucleic acid sequences encoding hypoglycosylated MUC-1 or immunogenic fragments thereof. The viral vector (e.g., an MVA vector) may be constructed using conventional techniques known to one of skill in the art. The one or more heterologous gene inserts encode a polypeptide having desired immunogenicity, i.e., a polypeptide that can induce an immune reaction, cellular immunity and/or humoral immunity, in vivo by administration thereof. The gene region of the viral vector (e.g., an MVA vector) where the gene encoding a polypeptide having immunogenicity is introduced is flanked by regions that are indispensable. In the introduction of a gene encoding a polypeptide having immunogenicity, an appropriate promoter may be operatively linked upstream of the gene encoding a polypeptide having desired immunogenicity.

In some embodiments, replication competent vaccinia viruses expressing MUC-1 peptides may be used to induce or boost an immune response to MUC-1. Vaccinia viruses have also been used to engineer viral vectors for recombinant gene expression and for the potential use as recombinant live vaccines (Mackett, M. et al 1982 PNAS USA 79:7415-7419; Smith, G. L. et al. 1984 Biotech Genet Engin Rev 2:383-407). This entails DNA sequences (genes) which code for foreign antigens being introduced, with the aid of DNA recombination techniques, into the genome of the vaccinia viruses. If the gene is integrated at a site in the viral DNA which is non-essential for the life cycle of the virus, it is possible for the newly produced recombinant vaccinia virus to be infectious, that is to say able to infect foreign cells and thus to express the integrated DNA sequence (EP Patent Applications No. 83,286 and No. 110,385). The recombinant vaccinia viruses prepared in this way can be used, on the one hand, as live vaccines for the prophylaxis of infectious diseases, on the other hand, for the preparation of heterologous proteins in eukaryotic cells.

In one embodiment, the nucleic acid sequence encodes an immunogenic extracellular domain sequence of MUC-1 and a transmembrane domain of the glycoprotein (GP) of Marburgvirus.

In one embodiment, the nucleic acid sequence encodes an immunogenic extracellular domain sequence of MUC-1 and a transmembrane domain of the glycoprotein (GP) of Marburgvirus and an intracellular domain sequence of MUC-1.

In one embodiment, the deletion III site is restructured and modified to remove non-essential flanking sequences.

In exemplary embodiments, the vaccine is constructed to express MUC-1, which is inserted between two conserved essential MVA genes (I8R and G1L) using shuttle vector pGeo-MUC-1; and to express MUC-1, which is inserted into deletion III using shuttle vector pGeo-MUC-1. pGeo-MUC-1 is constructed with an ampicillin resistance marker, allowing the vector to replicate in bacteria; with two flanking sequences, allowing the vector to recombine with a specific location in the MVA genome; with a green fluorescent protein (GFP) selection marker, allowing the selection of recombinant MVAs; with a sequence homologous to part of Flank 1 of the MVA sequence, enabling removal of the GFP sequence from the MVA vector after insertion of MUC-1 into the MVA genome; with a modified H5 (mH5) promoter, which enables transcription of the inserted MUC-1 sequence.

In certain embodiments, the polypeptide, or the nucleic acid sequence encoding the polypeptide, may have a mutation or deletion (e.g., an internal deletion, truncation of the amino- or carboxy-terminus, or a point mutation).

The one or more genes introduced into the recombinant viral vector are under the control of regulatory sequences that direct its expression in a cell.

The nucleic acid material of the viral vector may be encapsulated, e.g., in a lipid membrane or by structural proteins (e.g., capsid proteins), that may include one or more viral polypeptides.

In one embodiment, the sequence encoding a MUC-1 peptide or immunogenic fragment thereof is inserted into deletion site I, II, III, IV, V or VI of the MVA vector.

In one embodiment, the sequence encoding a MUC-1 peptide or immunogenic fragment thereof is inserted between I8R and G1L of the MVA vector, or into restructured and modified deletion III of the MVA vector; and a second sequence encoding a MUC-1 peptide or immunogenic fragment thereof is inserted between I8R and G1L of the MVA vector, or into restructured and modified deletion site III of the MVA vector.

In one embodiment, the recombinant vector comprises in a first deletion site, a nucleic acid sequence encoding a MUC-1 peptide or immunogenic fragment thereof operably linked to a promoter compatible with poxvirus expression systems, and in a second deletion site, a nucleic acid sequence encoding a VLP-forming protein operably linked to a promoter compatible with poxvirus expression systems.

In exemplary embodiments, the present invention is a recombinant MVA vector comprising at least one heterologous nucleic acid sequence (e.g., one or more sequences) encoding a MUC-1 peptide or immunogenic fragment thereof which is under the control of regulatory sequences that direct its expression in a cell. The sequence may be, for example, under the control of a promoter selected from the group consisting of Pm2H5, Psyn II, or mH5 promoters.

The recombinant viral vector of the present invention can be used to infect cells of a subject, which, in turn, promotes the translation into a protein product of the one or more heterologous sequence of the viral vector (e.g., a MUC-1 peptide or immunogenic fragment thereof). As discussed further herein, the recombinant viral vector can be administered to a subject so that it infects one or more cells of the subject, which then promotes expression of the one or more viral genes of the viral vector and stimulates an immune response that is therapeutic or protective against a neoplasm.

In one embodiment, the recombinant MVA vaccine expresses proteins that assemble into virus-like particles (VLPs) comprising the MUC-1 peptide or immunogenic fragment thereof. While not wanting to be bound by any particular theory, it is believed that the MUC-1 peptide is provided to elicit a protective immune response and the matrix protein is provided to enable assembly of VLPs and as a target for T cell immune responses, thereby enhancing the protective immune response and providing cross-protection.

In one embodiment, the matrix protein is a Marburg virus matrix protein.

In one embodiment, the matrix protein is an Ebola virus matrix protein.

In one embodiment, the matrix protein is a Sudan ebolavirus matrix protein.

In one embodiment, the matrix protein is a human immunodeficiency virus type 1 (HIV-1) matrix protein.

In one embodiment, the matrix protein is a human immunodeficiency virus type 1 (HIV-1) matrix protein encoded by the gag gene.

In one embodiment, the matrix protein is a Lassa virus matrix protein.

In one embodiment, the matrix protein is a Lassa virus Z protein.

In one embodiment, the matrix protein is a fragment of a Lassa virus Z protein.

In one embodiment, the matrix protein is a matrix protein of a virus in the Filoviridae virus family.

In one embodiment, the matrix protein is a matrix protein of a virus in the Retroviridae virus family.

In one embodiment, the matrix protein is a matrix protein of a virus in the Arenaviridae virus family.

In one embodiment, the matrix protein is a matrix protein of a virus in the Flaviviridae virus family.

One or more nucleic acid sequences may be optimized for use in an MVA vector. Optimization includes codon optimization, which employs silent mutations to change selected codons from the native sequences into synonymous codons that are optimally expressed by the host-vector system. Other types of optimization include the use of silent mutations to interrupt homopolymer stretches or transcription terminator motifs. Each of these optimization strategies can improve the stability of the gene, improve the stability of the transcript, or improve the level of protein expression from the sequence. In exemplary embodiments, the number of homopolymer stretches in the MUC-1 peptide sequence will be reduced to stabilize the construct. A silent mutation may be provided for anything similar to a vaccinia termination signal. An extra nucleotide may be added in order to express the transmembrane, rather than the secreted, form of any MUC-1 peptide.

In exemplary embodiments, the sequences are codon optimized for expression in MVA; sequences with runs of ≥5 deoxyguanosines, ≥5 deoxycytidines, ≥5 deoxyadenosines, and ≥5 deoxythymidines are interrupted by silent mutation to minimize loss of expression due to frame shift mutations; and the GP sequence is modified through addition of an extra nucleotide to express the transmembrane, rather than the secreted, form of the protein.

The present invention also extends to host cells comprising the recombinant viral vector described above, as well as isolated virions prepared from host cells infected with the recombinant viral vector.

### III. Pharmaceutical Composition

The recombinant viral vectors or immunogenic peptides described herein are readily formulated as pharmaceutical compositions for veterinary or human use, either alone or in combination. The pharmaceutical composition may comprise a pharmaceutically acceptable diluent, excipient, carrier, or adjuvant.

In one embodiment, the present invention is a vaccine effective to protect and/or treat a neoplasm comprising a recombinant MVA vector that expresses at least one MUC-1 peptide or an immunogenic fragment thereof. The vaccine composition may comprise one or more additional therapeutic agents.

The pharmaceutical composition may comprise 1, 2, 3, 4 or more than 4 different recombinant MVA vectors.

As used herein, the phrase “pharmaceutically acceptable carrier” encompasses any of the standard pharmaceutical carriers, such as those suitable for parenteral administration, such as, for example, by intramuscular, intraarticular (in the joints), intravenous, intradermal, intraperitoneal, and subcutaneous routes. Examples of such formulations include aqueous and non-aqueous, isotonic sterile injection solutions, which contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. One exemplary pharmaceutically acceptable carrier is physiological saline.

Other physiologically acceptable diluents, excipients, carriers, or adjuvants and their formulations are known to those skilled in the art.

In one embodiment, adjuvants are used as immune response enhancers. In various embodiments, the immune response enhancer is selected from the group consisting of alum-based adjuvants, oil based adjuvants, Specol, RIBI, TiterMax, Montanide ISA50 or Montanide ISA 720, GM-CSF, nonionic block copolymer-based adjuvants, dimethyl dioctadecyl ammoniumbromide (DDA) based adjuvants AS-1, AS-2, Ribi Adjuvant system based adjuvants, QS21, Quil A, SAF (Syntex adjuvant in its microfluidized form (SAF-m), dimethyl-dioctadecyl ammonium bromide (DDA), human complement based adjuvants m. vaccae, ISCOMS, MF-59, SBAS-2, SBAS-4, Enhanzyn®, RC-529, AGPs, MPL-SE, QS7, Escin; Digitonin; and Gypsophila, Chenopodium quinoa saponins

The compositions utilized in the methods described herein can be administered by a route selected from, e.g., parenteral, intramuscular, intraarterial, intravascular, intravenous, intraperitoneal, subcutaneous, dermal, transdermal, ocular, inhalation, buccal, sublingual, perilingual, nasal, topical administration, and oral administration. The preferred method of administration can vary depending on various factors (e.g., the components of the composition being administered, and the severity of the condition being treated). Formulations suitable for oral administration may consist of liquid solutions, such as an effective amount of the composition dissolved in a diluent (e.g., water, saline, or PEG-400), capsules, sachets or tablets, each containing a predetermined amount of the vaccine. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen).

For the purposes of this invention, pharmaceutical compositions suitable for delivering a therapeutic or biologically active agent can include, e.g., tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

The immunogenicity of the composition (e.g., vaccine) may be significantly improved if the composition of the present invention is co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM-Matrix, DC-Chol, DDA, cytokines, and other adjuvants and derivatives thereof.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Formulations suitable for oral administration can consist of (a) liquid solutions, such as an effective amount of the vaccine dissolved in diluents, such as water, saline or PEG 400; (b) capsules, sachets or tablets, each containing a predetermined amount of the vaccine, as liquids, solids, granules or gelatin; (c) suspensions in an appropriate liquid; (d) suitable emulsions; and (e) polysaccharide polymers such as chitins. The vaccine, alone or in combination with other suitable components, may also be made into aerosol formulations to be administered via inhalation, e.g., to the bronchial passageways. Aerosol formulations can be placed into pressurized acceptable propellants, such as dichlorodifluoromethane, propane, nitrogen, and the like.

Suitable formulations for rectal administration include, for example, suppositories, which consist of the vaccine with a suppository base. Suitable suppository bases include natural or synthetic triglycerides or paraffin hydrocarbons. In addition, it is also possible to use gelatin rectal capsules which consist of a combination of the vaccine with a base, including, for example, liquid triglycerides, polyethylene glycols, and paraffin hydrocarbons.

The vaccines of the present invention may also be co-administered with cytokines to further enhance immunogenicity. The cytokines may be administered by methods known to those skilled in the art, e.g., as a nucleic acid molecule in plasmid form or as a protein or fusion protein.

This invention also provides kits comprising the vaccines of the present invention. For example, kits comprising a vaccine and instructions for use are within the scope of this invention.

### IV. Method of Use

The compositions of the invention can be used as vaccines for inducing an immune response to hypoglycosylated MUC-1.

In exemplary embodiments, the present invention provides a method of inducing an immune response to MUC-1 peptide in a subject in need thereof, said method comprising administering a recombinant viral vector that encodes at least one MUC-1 peptide or immunogenic fragment thereof to the subject in an effective amount to generate an immune response to MUC-1 and a MUC-1 peptide in an effective amount to boost an immune response to MUC-1. The result of the method is that the subject is partially or completely immunized against the MUC-1 peptide.

In one aspect, the present invention provides a method of inducing an immune response to a neoplasm in a subject in need thereof, said method comprising:

a) administering a composition comprising an immunogenic vector expressing hypoglycosylated MUC-1 to the subject in an amount sufficient to induce an immune response, or boost a previously induced immune response and

b) administering a composition comprising a MUC-1 peptide in an amount sufficient to induce and immune response or boost a previously induced immune response.

In various embodiments, immunogenic fragments of MUC-1 may be expressed by the MVA vectors described herein or administered as peptide or peptide fragments to induce or boost an immune response to MUC-1.

In one embodiment, the MUC-1 peptide is an intracellular domain fragment of MUC-1.

In one embodiment, the MUC-1 peptide is an immunogenic intracellular domain fragment of MUC-1 (for example sequence 407-475 of GenBank Protein Accession Number NP_001191214 or an immunogenic fragment thereof).

In one embodiment, the MUC-1 peptide is an immunogenic extracellular domain fragment of MUC-1 (for example sequence 20-376 of GenBank Protein Accession Number NP_001191214 or an immunogenic fragment thereof).

In one embodiment, the MUC-1 peptide comprises the sequence TSAPDTRPAP (SEQ ID NO:1)

In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence

SEQ ID NO: 4)
AHGVTSAPDTRPAPGSTAPPAHGVTSAPDNRPALGSTAPP.

In one embodiment, the vectors express an extracellular domain fragment of MUC-1 consisting of the sequence

(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7)) wherein the threonine at position 27 is optionally glycosylated with alpha-D-GalNAc.

In one embodiment, the method comprises priming an immune response with an immunogenic vector expressing hypoglycosylated MUC-1 and boosting the immune response with a MUC-1 peptide.

In one embodiment, the immune response is a humoral immune response, a cellular immune response or a combination thereof.

In a particular embodiment, the immune response comprises production of binding antibodies to MUC-1.

In a particular embodiment, the immune response comprises production of neutralizing antibodies to MUC-1.

In a particular embodiment, the immune response comprises production of non-neutralizing antibodies to MUC-1.

In a particular embodiment, the immune response comprises production of a cell-mediated immune response to MUC-1.

In a particular embodiment, the immune response comprises production of neutralizing and non-neutralizing antibodies to MUC-1.

In a particular embodiment, the immune response comprises production of neutralizing antibodies and cell-mediated immunity to MUC-1.

In a particular embodiment, the immune response comprises production of non-neutralizing antibodies and cell-mediated immunity to MUC-1.

In a particular embodiment, the immune response comprises production of neutralizing antibodies, non-neutralizing antibodies, and cell-mediated immunity to MUC-1.

In one embodiment, the neoplasm is selected from leukemia (e.g. myeloblastic, promyelocytic, myelomonocytic, monocytic, erythroleukemia, chronic myelocytic (granulocytic) leukemia, and chronic lymphocytic leukemia), lymphoma (e.g. Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia.

In another embodiment, the neoplasm is selected from Adenocarcinomas (breast, colorectal, pancreatic, other), Carcinoid tumor, Chordoma, Choriocarcinoma, Desmoplastic small round cell tumor (DSRCT), Epithelioid sarcoma, Follicular dendritic cell sarcoma, interdigitating dendritic cell/reticulum cell sarcoma, Lung: type II pneumocyte lesions (type II cell hyperplasia, dysplastic type II cells, apical alveolar hyperplasia), Anaplastic large-cell lymphoma, diffuse large B cell lymphoma (variable), plasmablastic lymphoma, primary effusion lymphoma, Epithelioid mesotheliomas, Myeloma, Plasmacytomas, Perineurioma, Renal cell carcinoma, Synovial sarcoma (epithelial areas), Thymic carcinoma (often), Meningioma or Paget's disease.

In another aspect, the present invention provides a method of treating cancer comprising:

a) administering an effective amount of a recombinant MVA vector expressing hypoglycosylated MUC-1 to prime an immune response, and

b) a MUC-1 peptide in an effective amount to boost an immune response to a subject in need thereof to treat cancer.

In another aspect, the present invention provides a method of reducing growth of a neoplasm in a subject, said method comprising administering:

a) an effective amount of a recombinant MVA vector expressing hypoglycosylated MUC-1 to prime an immune response, and

b) a MUC-1 peptide in an effective amount to boost an immune response to a subject in need thereof to reduce growth of a neoplasm.

In another aspect, the present invention provides a method of reducing or preventing growth of a neoplasm in a subject, said method comprising administering:

a) an effective amount of a recombinant MVA vector expressing hypoglycosylated MUC-1 to prime an immune response, and

b) a MUC-1 peptide in an effective amount to boost an immune response to a subject in need thereof to reduce or prevent growth of a neoplasm in the subject.

In one embodiment, the subject expresses tumor cell markers, but not yet symptomatic. In a particular embodiment, treatment results in prevention of a symptomatic disease.

In another embodiment, the subject expresses tumor cell markers but exhibits minimal symptoms of cancer.

In another embodiment, the method results in amelioration of at least one symptom of cancer.

In one embodiment, invention provides methods for activating an immune response in a subject using the compositions described herein. In some embodiments, the invention provides methods for promoting an immune response in a subject using a composition described herein. In some embodiments, the invention provides methods for increasing an immune response in a subject using a composition described herein. In some embodiments, the invention provides methods for enhancing an immune response in a subject using a composition described herein.

In exemplary embodiments, the present invention provides a method of treating, reducing, preventing or delaying the growth of a neoplasm in a subject in need thereof, said method comprising administering the composition of the present invention to the subject in a therapeutically effective amount. The result of treatment is a subject that has an improved therapeutic profile for a disease associated with the neoplasm.

In exemplary embodiments, the present invention provides a method of treating, cancer in a subject in need thereof, said method comprising administering the composition of the present invention to the subject in a therapeutically effective amount. The result of treatment is a subject that has an improved therapeutic profile for a cancer.

In one embodiment the methods may reduce the growth of the one or more tumors, shrink the one or more tumors, or eradicate the one or more tumors. For example, the tumor mass does not increase. In certain embodiments, the tumor shrinks by 10%, 25%, 50%, 75%, 85%, 90%, 95%, or 99% or more (or any number therebetween) as compared to its original mass. In certain embodiments, the shrinkage is such that an inoperable tumor is sufficient to permit resection if desired. The concept of substantial shrinkage may also be referred to as “regression,” which refers to a diminution of a bodily growth, such as a tumor. Such a diminution may be determined by a reduction in measured parameters such as, but not limited to, diameter, mass (i.e., weight), or volume. This diminution by no means indicates that the size is completely reduced, only that a measured parameter is quantitatively less than a previous determination.

In one embodiment, the methods may prevent tumor metastasis.

In exemplary embodiments, the present invention provides a method of treating a proliferative disorder in a subject in need thereof, said method comprising administering the composition of the present invention to the subject in a therapeutically effective amount. As used herein, the term “proliferative disorder” refers to a disorder wherein the growth of a population of cells exceeds, and is uncoordinated with, that of the surrounding cells. In certain instances, a proliferative disorder leads to the formation of a tumor. In some embodiments, the tumor is benign, pre-malignant, or malignant. In other embodiments, the proliferative disorder is an autoimmune diseases, vascular occlusion, restenosis, atherosclerosis, or inflammatory bowel disease. In one embodiment, the autoimmune diseases to be treated may be selected from the group consisting of type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus), systemic lupus erythematosus (SLE), chronic polyarthritis, Basedow's disease, autoimmune forms of chronic hepatitis, colitis ulcerosa, allergy type I diseases, allergy type II diseases, allergy type III diseases, allergy type IV diseases, fibromyalgia, hair loss, Bechterew's disease, Crohn's disease, Myasthenia gravis, neuroclermitis, Polymyalgia rheumatica, progressive systemic sclerosis (PSS), psoriasis, Reiter's syndrome, rheumatic arthritis, psoriasis, vasculitis, etc, or type II diabetes.

In one embodiment, the immune response is a humoral immune response, a cellular immune response or a combination thereof.

In a particular embodiment, the immune response comprises production of binding antibodies against hypoglycosylated MUC-1.

In a particular embodiment, the immune response comprises production of neutralizing antibodies against hypoglycosylated MUC-1.

In a particular embodiment, the immune response comprises production of non-neutralizing antibodies against hypoglycosylated MUC-1.

In a particular embodiment, the immune response comprises production of a cell-mediated immune response against hypoglycosylated MUC-1.

In a particular embodiment, the immune response comprises production of neutralizing and non-neutralizing antibodies against hypoglycosylated MUC-1.

In a particular embodiment, the immune response comprises production of neutralizing antibodies and cell-mediated immunity against hypoglycosylated MUC-1.

In a particular embodiment, the immune response comprises production of non-neutralizing antibodies and cell-mediated immunity against hypoglycosylated MUC-1.

In a particular embodiment, the immune response comprises production of neutralizing antibodies, non-neutralizing antibodies, and cell-mediated immunity against hypoglycosylated MUC-1.

In certain embodiments, the compositions of the invention can be used as vaccines for treating a subject at risk of developing a neoplasm, or a subject already having a neoplasm. The recombinant viral vector comprises genes or sequences encoding MUC-1 and viral proteins to promote assembly of virus-like particles (VLPs) or additional enzymes to facilitate expression and glycosylation of hypoglycosylated MUC-1.

Typically, the vaccines will be in an admixture and administered simultaneously, but may also be administered separately.

A subject to be treated according to the methods described herein may be one who has been diagnosed by a medical practitioner as having such a condition. (e.g. a subject having a neoplasm). Diagnosis may be performed by any suitable means. One skilled in the art will understand that a subject to be treated according to the present invention may have been identified using standard tests or may have been identified, without examination, as one at high risk due to the presence of one or more risk factors.

Prophylactic treatment may be administered, for example, to a subject not yet having a neoplasm but who is susceptible to, or otherwise at risk of developing a neoplasm.

Therapeutic treatment may be administered, for example, to a subject already a neoplasm in order to improve or stabilize the subject's condition. The result is an improved therapeutic profile. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique.

For example, depending upon the type of cancer, an improved therapeutic profile may be selected from alleviation of one or more symptoms of the cancer, diminishment of extent of disease, stabilized (i.e., not worsening) state of disease, delay or slowing of disease progression, amelioration or palliation of the disease state, remission (whether partial or total), whether detectable or undetectable, tumor regression, inhibition of tumor growth, inhibition of tumor metastasis, reduction in cancer cell number, inhibition of cancer cell infiltration into peripheral organs, improved time to disease progression (TTP), improved response rate (RR), prolonged overall survival (OS), prolonged time-to-next-treatment (TNTT), or prolonged time from first progression to next treatment, or a combination of two or more of the foregoing.

In other embodiments, treatment may result in amelioration of one or more symptoms of a disease associated with a neoplasm (e.g. cancer). According to this embodiment, confirmation of treatment can be assessed by detecting an improvement in or the absence of symptoms.

In one embodiment, the present invention is a method of inducing an immune response in a subject (e.g., a human) by administering to the subject a recombinant viral vector that encodes at least one MUC-1 peptide or immunogenic fragment thereof. The immune response may be a cellular immune response or a humoral immune response, or a combination thereof.

The composition may be administered, e.g., by injection (e.g., intramuscular, intraarterial, intravascular, intravenous, intraperitoneal, or subcutaneous).

It will be appreciated that more than one route of administering the vaccines of the present invention may be employed either simultaneously or sequentially (e.g., boosting). In addition, the vaccines of the present invention may be employed in combination with traditional immunization approaches such as employing protein antigens, vaccinia virus and inactivated virus, as vaccines. Thus, in one embodiment, the vaccines of the present invention are administered to a subject (the subject is “primed” with a vaccine of the present invention) and then a traditional vaccine is administered (the subject is “boosted” with a traditional vaccine). In another embodiment, a traditional vaccine is first administered to the subject followed by administration of a vaccine of the present invention. In yet another embodiment, a traditional vaccine and a vaccine of the present invention are co-administered.

While not to be bound by any specific mechanism, it is believed that upon inoculation with a pharmaceutical composition as described herein, the immune system of the host responds to the vaccine by producing antibodies, both secretory and serum, specific for one or more MUC-1 peptides or immunogenic fragments thereof; and by producing a cell-mediated immune response specific for one or more MUC-1 peptides or immunogenic fragments thereof. As a result of the vaccination, the host becomes at least partially or completely immune to one or more MUC-1 peptides or immunogenic fragments thereof, or resistant to developing moderate or severe diseases caused by neoplasm.

In one aspect, methods are provided to alleviate, reduce the severity of, or reduce the occurrence of, one or more of the symptoms associated with a neoplasm comprising administering an effective amount of a pharmaceutical composition comprising a recombinant MVA viral vector that comprises a sequence encoding hypoglycosylated MUC-1, matrix protein sequences, and optionally co-expressing sequences that facilitate expression of and desired glycosylation the MUC-1 peptide.

In another aspect, the invention provides methods of providing anti-MUC-1 immunity comprising administering an effective amount of a pharmaceutical composition comprising a recombinant MVA vaccine expressing hypoglycosylated MUC-1 and a viral matrix protein to permit the formation of VLPs.

It will also be appreciated that single or multiple administrations of the vaccine compositions of the present invention may be carried out. For example, subjects who are at particularly high risk of developing a neoplasm may require multiple immunizations to establish and/or maintain protective immune responses. Levels of induced immunity can be monitored by measuring amounts of binding and neutralizing secretory and serum antibodies as well as levels of T cells, and dosages adjusted, or vaccinations repeated as necessary to maintain desired levels of protection.

In one embodiment, administration is repeated at least twice, at least 3 times, at least 4 times, at least 5 times, at least 6 times, at least 7 times, at least 8 times, or more than 8 times.

In one embodiment, administration is repeated twice.

In one embodiment, about 2-8, about 4-8, or about 6-8 administrations are provided.

In one embodiment, about 1-4-week, 2-4 week, 3-4 week, 1 week, 2 week, 3 week, 4 week or more than 4 week intervals are provided between administrations.

In one specific embodiment, a 4-week interval is used between 2 administrations.

In one embodiment, the invention provides a method of monitoring treatment progress. In exemplary embodiments, the monitoring is focused on biological activity, immune response and/or clinical response.

In one embodiment, the biological activity is a T-cell immune response, regulatory T-cell activity, molecule response (MRD), cytogenic response or conventional tumor response for example, in boht the adjuvant or advanced disease setting.

In one embodiment, immune response is monitored for example, by an immunse assay such as a cytotoxicity assay, an intracellular cytokine assay, a tetramer assay or an ELISPOT assay.

In one embodiment, clinical response is monitored for example by outcome using established definitions such as response (tumor regression), progression-free, recurrence free, or overall survival.

In one embodiment, the method includes the step of determining a level of diagnostic marker marker (e.g., any target delineated herein modulated by a compound herein, a protein or indicator thereof, etc.) or diagnostic measurement (e.g., screen, assay) in a subject having received a therapeutic amount of a compound herein sufficient to treat the disease or symptoms thereof. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to this invention; this pre-treatment level of marker can then be compared to the level of marker in the subject after the treatment commences, to determine the efficacy of the treatment.

In one embodiment, upon improvement of a subject's condition (e.g., a change (e.g., decrease) in the level of disease in the subject), a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

A. Combination with Checkpoint Inhibitors and Chemotherapy

In one embodiment, the above methods can further involve administering a standard of care therapy to the subject. In embodiments, the standard of care therapy is surgery, radiation, radio frequency, cryogenic, ultranoic ablation, systemic chemotherapy, or a combination thereof.

The vector compositions described herein may be provided as a pharmaceutical composition in combination with other active ingredients. The active agent may be, without limitation, including but not limited to radionuclides, immunomodulators, anti-angiogenic agents, cytokines, chemokines, growth factors, hormones, drugs, prodrugs, enzymes, oligonucleotides, siRNAs, pro-apoptotic agents, photoactive therapeutic agents, cytotoxic agents, chemotherapeutic agents, toxins, other antibodies or antigen binding fragments thereof.

In another embodiment, the pharmaceutical composition includes a MUC-1 peptide-expressing vector described herein and a checkpoint inhibitor to activate CD4+, CD8+ effector T-cells to increase tumor clearance.

In various embodiments, the checkpoint inhibitor is an antibody.

Antibodies are a key component of the adaptive immune response, playing a central role in both recognizing foreign antigens and stimulating an immune response. Many immunotherapeutic regimens involve antibodies. There are a number of FDA-approved antibodies useful as combination therapies. These antibodies may be selected from Alemtuzumab, Atezolizumab, Ipilimumab, Nivolumab, Ofatumumab, Pembrolizumab, or Rituximab.

Monoclonal antibodies that target either PD-1 or PD-L1 can boost the immune response against cancer cells and have shown a great deal of promise in treating certain cancers. Examples of antibodies that target PD-1 include Pembrolizumab and Nivolumab. An example of an antibody that targets PD-L1 is Atezolizumab.

CTLA-4 is another protein on some T cells that acts as a type of “off switch” to keep the immune system in check. Ipilimumab is a monoclonal antibody that attaches to CTLA-4 to block activity and boost an immune response against a neoplasm.

In another embodiment, the immunogenic vector compositions are administered with adjuvant chemotherapy to increase dendritic cell ability to induce T cell proliferation.

In various embodiments, the vector compositions are administered, before, after or at the same time as chemotherapy.

In certain embodiments, the composition of the present invention is able to reduce the need of a subject having a tumor or a cancer to receive chemotherapeutic or radiation treatment. In other embodiments, the composition is able to reduce the severity of side effects associated with radiation or chemotherapy in a subject having a tumor or cancer.

The pharmaceutical compositions of the present invention can be administered alone or in combination with other types of cancer treatment strategies (e.g., radiation therapy, chemotherapy, hormonal therapy, immunotherapy and anti-tumor agents as described herein.

Suitable chemotherapeutic agents useful with these methods include sorafenb, regorafenib, imatinib, eribulin, gemcitabine, capecitabine, pazopani, lapatinib, dabrafenib, sutinib malate, crizotinib, everolimus, torisirolimus, sirolimus, axitinib, gefitinib, anastrole, bicalutamide, fulvestrant, ralitrexed, pemetrexed, goserilin acetate, erlotininb, vemurafenib, visiodegib, tamoxifen citrate, paclitaxel, docetaxel, cabazitaxel, oxaliplatin, ziv-aflibercept, bevacizumab, trastuzumab, pertuzumab, pantiumumab, taxane, bleomycin, melphalen, plumbagin, camptosar, mitomycin-C, mitoxantrone, SMANCS, doxorubicin, pegylated doxorubicin, Folfori, 5-fluorouracil, temozolomide, pasireotide, tegafur, gimeracil, oteraci, itraconazole, bortezomib, lenalidomide, irintotecan, epirubicin, and romidepsin. Preferred chemotherapeutic agents are Carboplatin, Fluorouracil, Vinblastine, Gemcitabine, Cyclophosphamide, Doxorubicin, Methotrexate, Paclitaxel, Topotecan, Etoposide, Methotrexate, Sorafenib, Irinotecan, and Tarceva.

Generic names of cancer chemotherapeutic drugs that have been typically used in cancer patients include: doxorubicin, epirubicin; 5-fluorouracil, paclitaxel, docetaxel, cisplatin, bleomycin, melphalen, plumbagin, irinotecan, mitomycin-C, and mitoxantrone. By way of example, some other cancer chemotherapeutic drugs that may be used and may be in stages of clinical trials include: resminostat, tasquinimod, refametinib, lapatinib, Tyverb, Arenegyr, pasireotide, Signifor, ticilimumab, tremelimumab, lansoprazole, PrevOnco, ABT-869, linifanib, tivantinib, Tarceva, erlotinib, Stivarga, regorafenib, fluoro-sorafenib, brivanib, liposomal doxorubicin, lenvatinib, ramucirumab, peretinoin, Ruchiko, muparfostat, Teysuno, tegafur, gimeracil, oteracil, and orantinib.

Manufacturer brand names for some cancer drugs that may be used in the present invention include: NEXAVAR (sorafenb), STIVARGA (regorafenib), AFFINITOR (everolimus), GLEEVEC (imatinib), HALAVEN (eribulin), ALIMTA (pemetrexed), GEMZAR (gemcitabine), VOTRIENT (pazopanib), TYKERB (lapatinib), TAFINIAR (dabrafenib), SUTENT (sutinib malate), XALKORI (crizotinib), TORISEL (torisirolimus), INLYTA (axitinib), IRESSA (gefitinib), ARIMEDEX (anastrole), CASODEX (bicalutamide), FASLODEX (fulvestrant), TOMUDEX (ralitrexed), ZOLADEX (goserilin acetate), TARCEVA (erlotininb), XELODA (capecitabine), ZELBROF (vemurafenib), ERIVEDGE (visiodegib), PERJETA (pertuzumab), HERCEPTIN (trastuzumab), TAXOTERE (docetaxel), JEVTANA (cabazitaxel), ELOXATIN (oxaliplatin), ZALTRAP (ziv-aflibercept), AVASTIN (bevacizumab) Nolvadex, Istubal, and VALODEX (tamoxifen citrate), TEMODAR (temozolomide), SIGNIFOR (pasireotide), VECTIBIX (pantiumumab), ADRIAMYCIN (doxorubicin), DOXIL (pegylated doxorubicin), ABRAXANE (Paclitaxel), TEYSUNO (tegafur, gimeracil, oteracil), BORTEZOMIB (Velcade) and with lenalidomide, ISTODAX (romidepsin).

It is believed that one way that Doxorubicin (ADRIAMYCIN) and DOXIL (pegylated doxorubicin in liposomes) can act to kill cancer cells is by intercalating DNA. It is also thought that doxorubicin can become a nitroxide free radical and/or thereby increase cellular levels of free radicals in cancer cells and thereby trigger cellular damage and programmed death. There are potentially serious adverse systemic effects of doxorubicin such as heart damage which limit its use.

5-Fluorouracil (5-FU, Efudex) is a pyrimidine analog which is used in the treatment of cancer. It is a suicide inhibitor and works through irreversible inhibition of thymidylate synthase. Like many anti-cancer drugs, 5-FU's effects are felt system wide but fall most heavily upon rapidly dividing cells that make more frequent use of their nucleotide synthesis machinery, such as cancer cells. 5-FU kills non-cancer cells in parts of the body that are rapidly dividing, for example, the cells lining the digestive tract. Folfori is a treatment with 5-FU, Camptosar, and Irinotecan (leucovorin). The 5-FU incorporates into the DNA molecule and stops synthesis and Camptosar is a topoisomerase inhibitor, which prevents DNA from uncoiling and duplicating. Irinotecan (folinic acid, leucovorin) is a vitamin B derivative used as a “rescue” drug for high doses of the drug methotrexate and that modulates/potentiates/reduces the side effects of the 5-FU (fluorouracil). Mitomycin C is a potent DNA cross-linker. Prolonged use may result in permanent bone-marrow damage. It may also cause lung fibrosis and renal damage.

Taxanes agents include paclitaxel (Taxol) and docetaxel (Taxotere). Taxanes disrupt cell microtubule function. Microtubules are essential to cell division. Taxanes stabilize GDP-bound tubulin in the microtubule, thereby inhibiting the process of cell division. Cancer cells can no longer divide. However, taxanes may inhibit cell division of non-cancer cells as well.

Cisplatin(s) which includes carboplatin and oxaliplatin are organic platinum complexes which react in vivo, binding to and causing crosslinking of DNA. The cross-linked DNA triggers apoptosis (programmed cell death) of the cancer cells. However, cisplatins can also trigger apoptosis of non-cancer cells.

Bleomycin induces DNA strand breaks. Some studies suggest bleomycin also inhibits incorporation of thymidine into DNA strands. Bleomycin will also kill non-cancer cells. Melphalen (Alkeran) is a nitrogen mustard alkylating agent which adds an alkyl group to the guanine base of DNA. Major adverse effects of mephalen include vomiting, oral ulceration, and bone marrow suppression.

Plumbagin has been shown to induce cell cycle arrest and apoptosis in numerous cancer cell lines. It triggers autophagy via inhibition of the Akt/mTOR pathway. It induces G2/M cell cycle arrest and apoptosis through JNK-dependent p53 Ser15 phosphorylation. It promotes autophagic cell death. It inhibits Akt/mTOR signaling. It induces intracellular ROS generation in a PI 5-kinase-dependent manner. To non-cancer cells plumbagin is a toxin, a genotoxin, and a mutagen.

A chemotherapeutic agent may be selected based upon its specificity and potency of inhibition of a cellular pathway target to which cancer cells in the patient may be susceptible. In practicing the invention, the chemotherapeutic agent may be selected by its ability to inhibit a cellular pathway target selected from the group consisting of mTORC, RAF kinase, MEK kinase, Phosphoinositol kinase 3, Fibroblast growth factor receptor, multiple tyrosine kinase, Human epidermal growth factor receptor, vascular endothelial growth factor, other angiogenesis, heat shock protein; Smo (smooth) receptor, FMS-like tyrosine kinase 3 receptor, Apoptosis protein inhibitor, cyclin dependent kinases, deacetylase, ALK tyrosine kinase receptor, serine/threonine-protein kinase Pim-1, Porcupine acyltransferase, hedgehog pathway, protein kinase C, mDM2, Glypciin3, ChK1, Hepatocyte growth factor MET receptor, Epidermal growth factor domain-like 7, Notch pathway, Src-family kinase, DNA methyltransferase, DNA intercalators, Thymidine synthase, Microtubule function disruptor, DNA cross-linkers, DNA strand breakers, DNA alkylators, JNK-dependent p53 Ser15 phosphorylation inducer, DNA topoisomerase inhibitors, Bcl-2, and free radical generators.

In one embodiment, the vector compositions are administered, before, after or at the same time as epigenetic modulators.

In one embodiment, the vector compositions are administered, before, after or at the same time as an epigenetic modulator selected from the group consisting of inhibitors of DNA methyltransferases, inhibitors of histone methyltransferases, inhibitors of histone acetyltransferases, inhibitors of histone deacetylases, and inhibitors of lysine demethylases.

In one embodiment, the vector compositions are administered, before, after or at the same time as an inhibitor of DNA methyltransferases.

In one embodiment, the vector compositions are administered, before, after or at the same time as an inhibitor of histone deacetylases.

B. Dosage

The vaccines are administered in a manner compatible with the dosage formulation, and in such amount as will be therapeutically effective, immunogenic and protective. The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. However, suitable dosage ranges are readily determinable by one skilled in the art and generally range from about 5.0×10⁶ TCID₅₀ to about 5.0×10⁹ TCID₅₀. The dosage may also depend, without limitation, on the route of administration, the patient's state of health and weight, and the nature of the formulation.

The pharmaceutical compositions of the invention are administered in such an amount as will be therapeutically effective, immunogenic, and/or protective against a neoplasm that expresses a MUC-1 protein or fragment thereof. The dosage administered depends on the subject to be treated (e.g., the manner of administration and the age, body weight, capacity of the immune system, and general health of the subject being treated). The composition is administered in an amount to provide a sufficient level of expression that elicits an immune response without undue adverse physiological effects. Preferably, the composition of the invention is a heterologous viral vector that includes one MUC-1 peptide or immunogenic fragments thereof and large matrix protein; and is administered at a dosage of, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). A physician or researcher can decide the appropriate amount and dosage regimen.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition to the subject two or more times.

The term “effective amount” is meant the amount of a composition administered to improve, inhibit, or ameliorate a condition of a subject, or a symptom of a disorder, in a clinically relevant manner (e.g., improve, inhibit, or ameliorate disease associated with a neoplasm (e.g. cancer) or provide an effective immune response to a neoplasm). Any improvement in the subject is considered sufficient to achieve treatment. Preferably, an amount sufficient to treat is an amount that prevents the occurrence or one or more symptoms of disease associated with a neoplasm or is an amount that reduces the severity of, or the length of time during which a subject suffers from, one or more symptoms of disease associated with a neoplasm (e.g., by at least 10%, 20%, or 30%, more preferably by at least 50%, 60%, or 70%, and most preferably by at least 80%, 90%, 95%, 99%, or more, relative to a control subject that is not treated with a composition of the invention). A sufficient amount of the pharmaceutical composition used to practice the methods described herein (e.g., the treatment of disease associated with a neoplasm) varies depending upon the manner of administration and the age, body weight, and general health of the subject being treated.

It is important to note that the value of the present invention may never be demonstrated in terms of actual clinical benefit. Instead, it is likely that the value of the invention will be demonstrated in terms of success against a surrogate marker for protection. For an indication such as disease associated with a neoplasm, in which it is impractical or unethical to attempt to measure clinical benefit of an intervention, the FDA's Accelerated Approval process allows approval of a new vaccine based on efficacy against a surrogate endpoint. Therefore, the value of the invention may lie in its ability to induce an immune response that constitutes a surrogate marker for protection.

Similarly, FDA may allow approval of vaccines against hypoglycosylated MUC-1 based on its Animal Rule. In this case, approval is achieved based on efficacy in animals.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition two or more times.

In some instances it may be desirable to combine the MUC-1 vaccine of the present invention with vaccines which induce protective responses to other agents, particularly other MUC-1 peptides. For example, the vaccine compositions of the present invention can be administered simultaneously, separately or sequentially with other genetic immunization vaccines such as those for influenza (Ulmer, J. B. et al., Science 259:1745-1749 (1993); Raz, E. et al., PNAS (USA) 91:9519-9523 (1994)), malaria (Doolan, D. L. et al., J. Exp. Med. 183:1739-1746 (1996); Sedegah, M. et al., PNAS (USA) 91:9866-9870 (1994)), and tuberculosis (Tascon, R. C. et al., Nat. Med. 2:888-892 (1996)).

C. Administration

As used herein, the term “administering” refers to a method of giving a dosage of a pharmaceutical composition of the invention to a subject. The compositions utilized in the methods described herein can be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, intraarterial, intravascular, and intramuscular administration. The preferred method of administration can vary depending on various factors (e.g., the components of the composition being administered, and the severity of the condition being treated).

Administration of the pharmaceutical compositions (e.g., vaccines) of the present invention can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered, and the severity of the condition being treated.

In addition, single or multiple administrations of the compositions of the present invention may be given to a subject. For example, subjects who are particularly susceptible to developing a neoplasm may require multiple treatments to establish and/or maintain protection against the neoplasm. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against development of a neoplasm or to reduce growth of a neoplasm.

Increased vaccination efficacy can be obtained by timing the administration of the vector. Any of the priming and boosting compositions described above are suitable for use with the methods described here.

In one embodiment, recombinant MUC-1 expressing MVA vectors are administered to prime an immune response and the primed immune response is boosted at a time after the first MVA administration.

In one embodiment, a MUC-1-expressing MVA vector is administered to prime an immune response and a composition comprising a MUC-1-expressing MVA, a MUC-1 peptide, and/or a checkpoint inhibitor is administered to boost the immune response primed with the MUC-1-expressing MVA vector.

In one embodiment, MVA vectors are used for both priming and boosting purposes. Such protocols include but are not limited to MM, MMM, and MMMM.

In some embodiments, one, two, three, four, five, six, seven, eight, nine, ten or more than ten MVA or MUC-1 peptide boosts are administered.

In some embodiments, one, two, three, four, five, six, seven, eight, nine, ten or more than ten doses of checkpoint inhibitor are administered.

Vectors can be administered alone (i.e., a plasmid can be administered on one or several occasions with or without an alternative type of vaccine formulation (e.g., with or without administration of protein or another type of vector, such as a viral vector)) and, optionally, with an adjuvant or in conjunction with (e.g., prior to) an alternative booster immunization (e.g., a live-vectored vaccine such as a recombinant modified vaccinia Ankara vector (MVA)) comprising an insert that may be distinct from that of the “prime” portion of the immunization or may be a related vaccine insert(s). For example, GM-CSF or other adjuvants known to those of skill in the art. The adjuvant can be a “genetic adjuvant” (i.e., a protein delivered by way of a DNA sequence).

In exemplary embodiments, the present invention is an immunization method comprising (i) administering a priming composition comprising a MVA vector comprising one or more sequences encoding a hypoglycosylated MUC-1 or immunogenic fragment thereof; (ii) administering a first dose of a boosting composition comprising a hypoglycosylated MUC-1 peptide or immunogenic fragment thereof.

In a particular embodiment, the hypoglycosylated MUC-1 peptides are the same in step (i)-(iii). Optionally, the method further comprises one or more additional steps, including, for example, the administration of one or more additional doses of the priming composition or a different priming composition (i.e., a second priming composition) and/or one or more additional doses of the boosting composition or a different boosting composition (i.e., a second boosting composition).

D. Indications

In specific embodiments, the immunogenic vectors useful in the present methods may be administered to a subject with a neoplasm or a subject diagnosed with prostate, breast, lung, liver, endometrial, bladder, colon or cervical carcinoma; adenocarcinoma; melanoma; lymphoma; glioma; or sarcomas such as soft tissue and bone sarcomas

In a further embodiment the invention is directed to the vectors of the invention for the treatment or prevention of cancer, including, but not limited to, neoplasms, tumors, metastases, or any disease or disorder characterized by uncontrolled cell growth, and particularly multidrug resistant forms thereof. The cancer can be a multifocal tumor. Examples of types of cancer and proliferative disorders to be treated with the therapeutics of the invention include, but are not limited to, leukemia (e.g. myeloblastic, promyelocytic, myelomonocytic, monocytic, erythroleukemia, chronic myelocytic (granulocytic) leukemia, and chronic lymphocytic leukemia), lymphoma (e.g. Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to patients having prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). In an especially preferred embodiment the medicaments of the present invention are used for the treatment of cancer, glioma, liver carcinoma and/or colon carcinoma. The treatment and/or prevention of cancer includes, but is not limited to, alleviating symptoms associated with cancer, the inhibition of the progression of cancer, the promotion of the regression of cancer, and the promotion of the immune response.

As used herein, the term neoplasm refers to an abnormal growth of tissue. A neoplasm may be benign or malignant. Generally, a malignant neoplasm is referred to as a cancer. Cancers differ from benign neoplasms in the ability of malignant cells to invade other tissues, either by direct growth into adjacent tissue through invasion or by implantation into distant sites by metastasis (i.e., transport through the blood or lymphatic system). The methods of the present invention are suitable for the treatment of benign and malignant neoplasms (cancer).

As defined herein a superficial neoplasm is one located on the outer surface of the body that has confined itself and not spread to surrounding tissues or other parts of the body. An internal neoplasms located on an internal organ or other internal part of the body. An invasive neoplasm is a neoplasm that has started to break through normal tissue barriers and invade surrounding areas, e.g., an invasive breast cancer that has spread beyond the ducts and lobules

A non-exclusive list of the types of neoplasms contemplated for treatment by the method disclosed herein includes the following categories: (a) abdominal neoplasms including peritonealneoplasms and retroperitoneal neoplasms; (b) bone neoplasms including femoral neoplasms, skull neoplasms, jaw neoplasms, manibular neoplasms, maxillary neoplasms, palatal neoplasms, nose neoplasms, orbital neoplasms, skull base neoplasms, and spinal neoplasms; c) breast neoplasms including male breast neoplasms, breast ductal carcinoma, and phyllodes tumor; (d) digestive system neoplasms including biliary tract neoplasms, bile duct neoplasms, common bile duct neoplasms, gall bladder neoplasms, gastrointestinal neoplasms, esophegeal neoplasms, intestinal neoplasms, cecal neoplasms, appendiceal neoplasms, colorectal neoplasms, colorectal adenomatous polyposis coli, colorectal Gardner Syndrome, colonic neoplasms, colonic adenomatous polyposis coli, colonic Gardner Syndrome, sigmoid neoplasms, hereditary nonpolyposis colorectal neoplasms, rectal neoplasms, anus neoplasms, duodenal neoplasms, ileal neoplasms, jejunal neoplasms, stomach neoplasms, liver neoplasms, liver cell adenoma, hepatocellular carcinoma, pancreatic neoplasms, islet cell adenoma, insulinoma, islet cell carcinoma, gastrinoma, glucagonoma, somatostatinoma, vipoma, pancreatic ductal carcinoma, and peritoneal neoplasms; (e) endocrine gland neoplasms including adrenal gland neoplasms, adrenal cortex neoplasms, adrenocortical adenoma, adrenocortical carcinoma, multiple endocrine neoplasia, multiple endocrine neoplasia type 1, multiple endocrine neoplasia type 2a, multiple endocrine neoplasia type 2b, ovarian neoplasms, granulosa cell tumor, luteoma, Meigs' Syndrome, ovarian Sertoli-Leydig cell tumor, thecoma, pancreatic neoplasms, paraneoplastic endocrine syndromes, parathyroid neoplasms, pituitary neoplasms, Nelson Syndrome, testicular neoplasms, testicular Sertoli-Leydig cell tumor, and thyroid neoplasms (f) eye neoplasms including conjunctival neoplasms, orbital neoplasms, retinal neoplasms, retinoblastoma, uveal neoplasms, choroid neoplasms, and iris neoplasms; (g) brain, head and neck neoplasms including esophageal neoplasms, facial neoplasms, eyelid neoplasms, mouth neoplasms, gingival neoplasms, oral leukoplakia, hairy leukoplakia, lip neoplasms, palatal neoplasms, salivary gland neoplasms, parotid neoplasms, sublingual gland neoplasms, submandibular gland neoplasms, tongue neoplasms, otorhinolaryngologic neoplasms, ear neoplasms, laryngeal neoplasms, nose neoplasms, paranasal sinus neoplasms, maxillary sinus neoplasms, pharyngeal neoplasms, hypopharyngeal neoplasms, nasopharyngeal neoplasms, nasopharyngeal neoplasms, oropharyngeal neoplasms, tonsillar neoplasms, parathyroid neoplasms, thyroid neoplasms, and tracheal neoplasms; (h) hematologic neoplasms including bone marrow neoplasms; (i) nervous system neoplasms including central nervous system neoplasms, brain neoplasms, cerebral ventricle neoplasms, choroid plexus neoplasms, choroid plexus papilloma, infratentorial neoplasms, brain stem neoplasms, cerebellar neoplasms, neurocytoma, pinealoma, supratentorial neoplasms, hypothalamic neoplasms, pituitary neoplasms, Nelson Syndrome, cranial nerve neoplasms, optic nerve neoplasms, optic nerve glioma, acoustic neuroma, neurofibromatosis 2, nervous system paraneoplastic syndromes, Lambert-Eaton myasthenic syndrome, limbic encaphalitis, transverse myelitis, paraneoplastic cerebellar degeneration, paraneoplastic polyneuropathy, peripheral nervous system neoplasms, cranial nerve neoplasms, acoustic neuroma, and optic nerve neoplasms; (j) pelvic neoplasms; (k) skin neoplasms including acanthoma, sebaceous gland neoplasms, sweat gland neoplasms and basal cell carcinoma; (l) soft tissue neoplasms including muscle neoplasms and vascular neoplasms; (m) splenic neoplasms; (n) thoracic neoplasms including heart neoplasms, mediastinal neoplasms, respiratory tract neoplasms, bronchial neoplasms, lung neoplasms, bronchogenic carcinoma, non-small-cell lung carcinoma, pulmonary coin lesion, Pancoasts's Syndrome, pulmonary blastoma, pulmonary sclerosing hemangioma, pleural neoplasms, malignant pleural effusion, tracheal neoplasms, thymus neoplasms, and thymoma; (o) urogenital neoplasms including female genital neoplasms, fallopian tube neoplasms, uterine neoplasms, cervix neoplasms, endometrial neoplasms, endometrioid carcinoma, endometrial stromal tumors, endometrial stromal sarcoma, vaginal neoplasms, vulvar neoplasms, male genital neoplasms, penile neoplasms, prostatic neoplasms, testicular neoplasms, urologic neoplasms, bladder neoplasms, kidney neoplasms, renal cell carcinoma, nephroblastoma, Denys-Drash Syndrome, WAGR Syndrome, mesoblastic nephroma, ureteral neoplasms and urethral neoplasms; (p) and additional cancers including renal carcinoma, lung cancer, melanoma, leukemia, Barrett's esophagus, metaplasia pre-cancer cells.

In one embodiment, the immune response stimulating vectors described herein express MUC-1 or an immunogenic fragment thereof and are particularly useful for treating Adenocarcinomas (breast, colorectal, pancreatic, other), Carcinoid tumor, Chordoma, Choriocarcinoma, Desmoplastic small round cell tumor (DSRCT), Epithelioid sarcoma, Follicular dendritic cell sarcoma, interdigitating dendritic cell/reticulum cell sarcoma, Lung: type II pneumocyte lesions (type II cell hyperplasia, dysplastic type II cells, apical alveolar hyperplasia), Anaplastic large-cell lymphoma, diffuse large B cell lymphoma (variable), plasmablastic lymphoma, primary effusion lymphoma, Epithelioid mesotheliomas, Myeloma, Plasmacytomas, Perineurioma, Renal cell carcinoma, Synovial sarcoma (epithelial areas), Thymic carcinoma (often), Meningioma or Paget's disease.

The claimed invention is further described by way of the following non-limiting examples. Further aspects and embodiments of the present invention will be apparent to those of ordinary skill in the art, in view of the above disclosure and following experimental exemplification, included by way of illustration and not limitation, and with reference to the attached figures.

### EXAMPLES

### Example 1: MVA Vaccine Construction and In Vitro Evaluation for Hypoglycosylated Forms of MUC-1

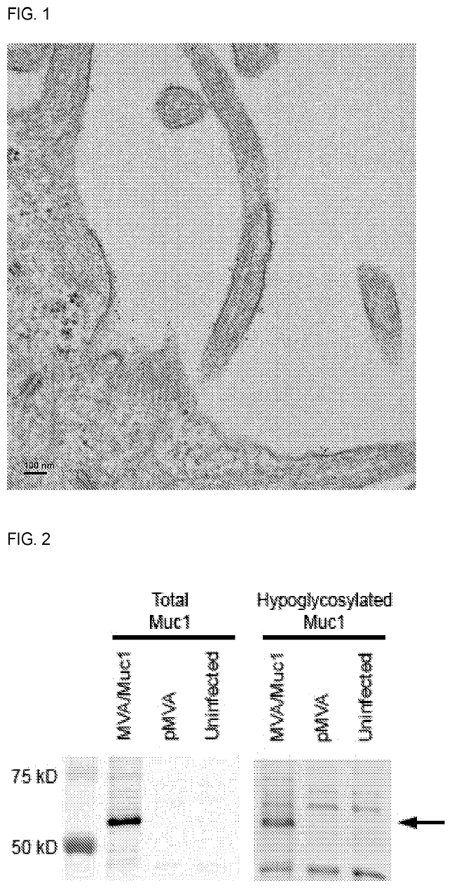

The recombinant MVA vaccine consists of an MVA vector with two antigen expression cassettes (MVA-MUC-1VP40). One expression cassette encodes a chimeric form of human MUC-1, the construction of which is described in WO 2017/120577 (hereafter this construction is called GVX-MUC-1) and which for the purposes of MVA vaccine construction has had its DNA sequence cloned into a shuttle plasmid entitled pGeo-MUC-1 (image of plasmid is seen above). One expression cassette encodes the VP40 protein of Marburgvirus. The expression of GVX-MUC-1 and VP40 is sufficient to generate secreted virus-like particles (VLPs). The GVX-MUC-1 protein is expressed as a chimeric protein consisting of the extracellular domain of human MUC-1, the transmembrane domain of Marburgvirus GP, and the intracellular domain of human MUC-1. Marburg VP40 protein is expressed in the cytoplasm of the cells where it associates with the intracellular domain and transmembrane domain of the GVX-MUC-1, causing cell-surface budding of VLPs that have GVX-MUC-1 on their surface and VP40 enclosed in their interior (luminal) space. This novel combination of vector platform and native antigen conformation yields a vaccine that is expected to elicit a strong, broad, and durable immune response. The MVA-MUC-1-VP40 vaccine candidate was constructed using shuttle vectors developed in the laboratory of Dr. Bernard Moss and are being licensed by the NIAID to GeoVax for use in vaccine development. These shuttle vectors have proven to yield stable vaccine inserts with high, but non-toxic, levels of expression in our work with HIV and hemorrhagic fever virus vaccines. The MUC-1 sequence was placed between two essential genes of MVA (I8R and G1L) and VP40 was inserted into a restructured and modified deletion III between the A50R and B1R genes, illustrated in the following schematic (FIG. 2), wherein the numbers refer to coordinates in the MVA genome:

The GVX-MUC-1 and VP40 genes were codon optimized for MVA. Silent mutations have been introduced to interrupt homo-polymer sequences (>4G/C and >4A/T) to reduce RNA polymerase errors that could lead to frameshifts. Inserted sequences have been edited for vaccinia-specific terminators to remove motifs that could lead to premature termination. All vaccine inserts are placed under the modified H5 early/late vaccinia promoter as described previously. Vectors were being prepared in a dedicated room under “GLP-like conditions” at GeoVax, with full traceability and complete documentation of all steps using Bovine Spongiform Encephalopathy/Transmissible Spongiform Encephalopathy (BSE/TSE)-free raw materials.

The expression of full length and native conformation of GVX-MUC-1 protein expressed in cells were assessed by western blotting using MUC-1-specific antibodies. The MVA-MUC-1VP40 vaccine was used to infect DF1 cells at a multiplicity of infection of 1.0 for 1 hour at 37° C. after which time the medium was exchanged for fresh pre-warmed medium. After 48 hours incubation at 37° C. the supernatant of the cells was harvested and clarified by centrifuging at 500×g for 10 minutes. Once the supernatant was removed from the cells, the cells themselves were harvested from the plate, washed once with cold phosphate-buffered saline (PBS) and were then lysed on ice for 15 minutes in a solution of PBS+1% Triton X-100 detergent. After this incubation, a post-nuclear supernatant was prepared by centrifuging the lysate at 1000×g for 10 minutes and harvesting the liquid layer on top, which is hereafter termed the “cell lysate”. The cell lysates were applied to 10% SDS-PAGE gels and were separated by electrophoresis, then transferred to nitrocellulose membranes, blocked with Odyssey blocking buffer, then incubated with a primary antibody that recognizes either (1) the total amount of MUC-1 present in the sample, or (2) the total amount of hypoglycosylated MUC-1 in the sample. As control, supernatant and cell lysate from DF1 cells infected with parental MVA (a vector control containing none of the antigen expression cassettes). The results of this analysis are seen in the following image of the western blot (FIG. 2):

This demonstrates that the MVA-MUC-1VP40 vaccine infects DF1 cells and expresses MUC-1 protein and furthermore demonstrates that some proportion of the MUC-1 expressed is in hypoglycosylated form.

Evidence of the hypoglycosylated form of MUC-1 encoded by the MVA-MUC-1VP40 vaccine is seen by immunostaining cells infected with the vaccine or simultaneously staining control cells that are known to express either normally-glycosylated or hypo-glycosylated MUC-1, as described here:
Control cell lines MCF7 and MCF10A both express MUC-1. 293T cells do not.
MCF7 cell express hypo-glycosylated MUC-1, recognized by a hypoglycosylated MUC-1-specific Ab (4H5).
MCF10A expresses normal MUC-1. A pan-MUC-1 Ab (HMPV) is used to detect total MUC-1.
293T cells were infected with MVA-MUC-1VP40 or MVA control virus (parental MVA).
All samples were stained with the indicated Abs.

VLP formation was shown by immune-electron microscopy (EM) using of DF1 cells infected with the MVA-MUC-1-VP40 vaccine and stained with a monoclonal antibody that recognizes MUC-1 (HMPV). In the EM image below (FIG. 1) two thing are clearly illustrated: (1) that the VLPs are filamentous, a phenomenon derivative of the fact that the VP40 protein is used as the matrix protein that drive VLP budding from the surface of cells; and (2) that the VLPs stain positively with the antibody directed against MUC-1, demonstrating that this protein is incorporated into the budding VLPs.

### Example 2: Assessment of Induction of Anti-Tumor MUC-1 T and B Cell Responses in Non-Tumor Bearing hMUC-1 Transgenic Mice Using MTI and/or MVA-MUC-1-VP40

TABLE 1
Experiment 1 treatment groups. 3 mice per group, 7 groups, 21 mice total.

| Group | Treatment | d 0 | d 7 | d 14 | d 21 | d 28 | d 35 |
|---|---|---|---|---|---|---|---|
| 1 | Control | — | — | — | — | — | Analyze |
| 2 | MTI | MTI | MTI | MTI | MTI | — | Analyze |
| 3 | MTI | MTI | — | — | MTI | — | Analyze |
| 4 | MVA | MVA | — | — | MVA | — | Analyze |
| 5 | MVA > MTI | MVA | — | — | MTI | — | Analyze |
| 6 | MTI > MVA | MTI | — | — | MVA | — | Analyze |
| 7 | MVA + MTI | MVA + MTI | — | — | MVA + MTI | — | Analyze |

| | |
|---|---|
| Collect | Collect |
| sera | sera |

i. Analysis

Two weeks after the last immunization (day 35), the mice are sacrificed. Splenocytes are harvested and sera is collected.

Data:

Antibody ELISAs:

Humoral immune responses are assessed by measuring titers of MUC-1-specific antibodies using ELISA. ELISA plates were coated with BSA conjugated to TSAPDT(aGalNAc)RPAP, to TSAPDTRPAP, or unconjugated BSA.

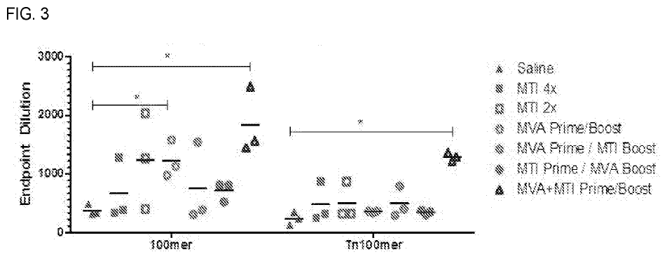

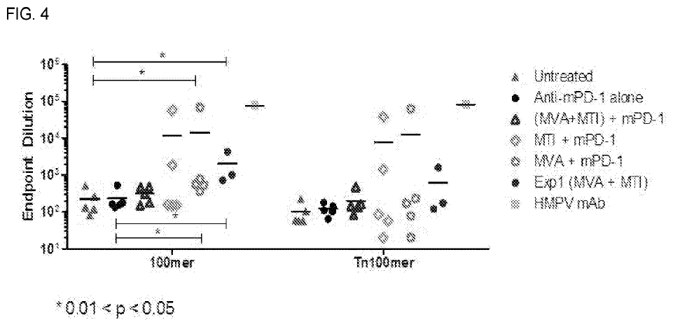

Results are shown in Table 1 and FIG. 3.

| | Control | | | MTI 4× | | | MTI 2× | | | MVA | | |
|---|---|---|---|---|---|---|---|---|---|---|---|---|
| Cage Number | 1 | 10 | 15 | 4 | 11 | 2 | 5 | 3 | 18 | 12 | 14 | 8 |
| Day 14 MUC(Tn) | | | | 1366 | | | 0 | | | 0 | | |
| Day 35 MUC (Tn) | 0 | 0 | 0 | 17889 | 2825 | 2351 | 5050 | 1738 | 23524 | 0 | 0 | 0 |
| Day 14 Unglyc | | | | 1535 | | | 0 | | | 0 | | |
| MUC | | | | | | | | | | | | |
| Day 35 Unglyc | 0 | 0 | 0 | 18467 | 2995 | 2147 | 5307 | 1313 | 11644 | 0 | 0 | 0 |
| MUC | | | | | | | | | | | | |

| | | | | | | | | | | Exp 16 Mayo 2012 | | |
|---|---|---|---|---|---|---|---|---|---|---|---|---|
| | MVA > MTI | | | MTI > MVA | | | MVA + MTI | | | (4× bi-weekly) | | |
| Cage Number | 9 | 13 | 7 | 16 | 21 | 17 | 20 | 19 | 6 | 1b | 1d | 1f |
| Day 14 MUC(Tn) | | | | | | | 0 | | | | | |
| Day 35 MUC (Tn) | 0 | 0 | 0 | 618 | 2569 | 0 | 3291 | 732 | 372 | 45983 | 7442 | 40928 |
| Day 14 Unglyc | | | | | | | 0 | | | | | |
| MUC | | | | | | | | | | | | |
| Day 35 Unglyc | 0 | 299 | 0 | 578 | 2445 | 0 | 3288 | 890 | 0 | 32349 | 6866 | 30270 |
| MUC | | | | | | | | | | | | |

T Cell Analysis:

MUC-1-specific CD8 and CD4 immune responses were assessed by intracellular staining (ICS). Splenocytes were stimulated in vitro with TA-MUC-1 TSAPDT(GalNAc)RPAP, unglycosylated MUC-1 (TSAPDTRPAP) and non-MUC-1 peptides (HIV-1 Env peptides, negative control) and a MUC-1 peptide library prior to ICS

| Vaccination | | IFNg + Animals | | TNF + Animals | |
|---|---|---|---|---|---|
| Condition | Muc 1 Peptide | CD4+ | CD8+ | CD4+ | CD8+ |
| Saline | Short, non-g | 0 | 0 | 0 | 0 |
| | Short, g | 0 | 0 | 0 | 0 |
| | Long, g | 0 | 0 | 0 | 0 |
| | Muc 1 Library | 0 | 0 | 0 | 0 |
| MTI (4 dose) | Short, non-g | 1 | 1 | 3 | 0 |
| | Short, g | 0 | 0 | 0 | 0 |
| | Long, g | 1 | 1 | 0 | 0 |
| | Muc 1 Library | 0 | 0 | 0 | 0 |
| MTI (2 dose) | Short, non-g | 0 | 0 | 1 | 0 |
| | Short, g | 0 | 0 | 0 | 0 |
| | Long, g | 0 | 0 | 0 | 0 |
| | Muc 1 Library | 0 | 0 | 0 | 0 |
| MVA | Short, non-g | 1 | 1 | 0 | 0 |
| | Short, g | 1 | 1 | 0 | 0 |
| | Long, g | 1 | 1 | 0 | 0 |
| | Muc 1 Library | 0 | 0 | 0 | 0 |
| MVA −> MTI | Short, non-g | 1 | 1 | 0 | 0 |
| | Short, g | 1 | 1 | 1 | 1 |
| | Long, g | 0 | 0 | 0 | 0 |
| | Muc 1 Library | 0 | 0 | 0 | 0 |
| MTI −> MVA | Short, non-g | 0 | 0 | 0 | 0 |
| | Short, g | 0 | 0 | 0 | 0 |
| | Long, g | 1 | 1 | 0 | 0 |
| | Muc 1 Library | 0 | 0 | 0 | 0 |
| MVA + MTI | Short, non-g | 0 | 0 | 1 | 0 |
| | Short, g | 0 | 1 | 0 | 0 |
| | Long, g | 0 | 0 | 0 | 0 |
| | Muc 1 Library | 0 | 0 | 0 | 0 |

| | | | | |
|---|---|---|---|---|
| Heatmap Key (animal #): | 0 | 0 | 3 | 3 |

| | | |
|---|---|---|
| Muc 1 Peptide Name Key: | Name | Description |
| | Short, non-g | Short Muc 1 Peptide, non-glycosylated |
| | Short, g | Short Muc 1 Peptide, glycosylated |
| | Long, g | Long Muc 1 Peptide, glycosylated |
| | Muc 1 Library | Full Muc 1 Sequence Peptide Library |

### Example 3: Assessment and Optimization of a Combined MUC-1 Vaccine and Immune Checkpoint Inhibitor Therapy to Effect Tumor Regression in Mice with Established MUC-1+ Tumors Using the Therapeutic hMUC-1Tg Mouse Tumor Model

Compositions of MTI, MVA and MTI+MVA were evaluated for ability to enhance anti-tumor activity of anti-PD-1 antibody. MC38 MUC-1 cells (implanted SC) will be used for the experiment.

| Treatment | | |
|---|---|---|
| Group | MUC-1 | Anti-mPD-1 |
| 1 | | |
| 2 | | yes |
| 3 | MTI (4 dose) | yes |
| 4 | MVA (2 dose) | yes |
| 5 | MTI (2 dose) + MVA (2 dose) | yes |

hMUC-1 Tg mice

hMUC-1 MC38 tumor cells

Anti-mPD-1 dosed 2× per week for 5 wks, starting on d8.

5 mice per group

Tumor calculated from caliper measurements.

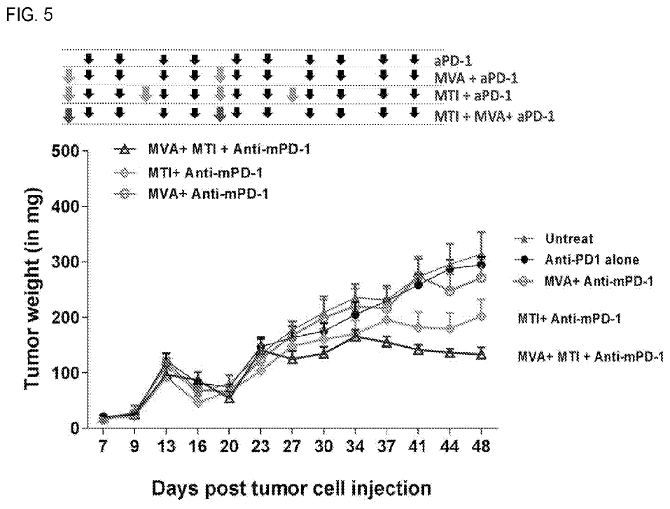

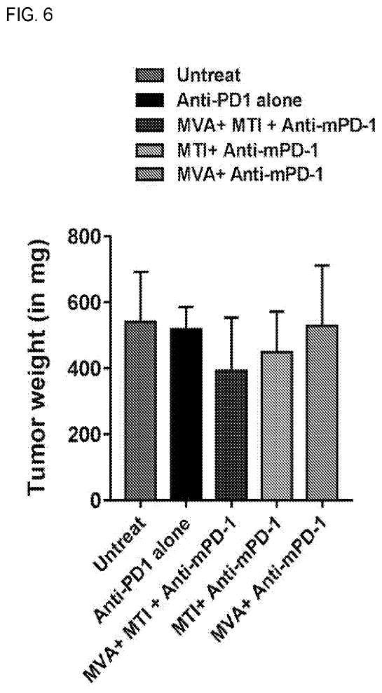

Results are shown in FIGS. 5 and 6.

### Example 4: Assessment of Induction of Anti-Tumor MUC-1 T and B Cell Responses in Non-Tumor Bearing hMUC-1 Transgenic Mice Using Tn-100-Mer (Tn-MUC-1) and/or MVA-MUC-1VP40

For the MVA-VLP-MUC-1 vaccine, the cell surface protein recombined into the MVA genome is the gene for human MUC-1, a highly glycosylated type-1 transmembrane protein expressed on the apical membrane of epithelial cells. Human MUC-1 associated with transformed cells is expressed in a hypo-glycosylated form, acting as a cancer neo-antigen that results from aberrant post-translation modification of the protein. The MUC-1 incorporated into the MVA-VLP-MUC-1 vaccine was modified in such a way that the MUC-1 transmembrane domain (TM) was swapped out with the TM of MARV GP protein. The MUC-1 gene was placed behind the modified H5 promoter of Vaccinia, facilitating a moderate-to-high level of expression in infected cells.

In addition to MUC-1, the MVA-VLP-MUC-1 cancer vaccine expresses the VP40 gene from Marburg virus. Expressed VP40 associates with (i) the inner leaflet of the plasma membrane, (ii) the TM of MARV GP, and (iii) itself in a polymeric form, all of which facilitate the budding of VLPs from the surface of the infected cell. Because the MUC-1 incorporated into the MVA-VLP-MUC-1 vaccine contains the TM of MARV GP, it is MUC-1 that directly associates with VP40, facilitating the production of VLPs that bear MUC-1 on their surface.

Infection of cells with MVA-VLP-MUC-1 drives expression of both MUC-1 and VP40 from the cells. Importantly, by using antibodies (Abs) that are specific for hypo-glycosylated MUC-1, consistent reactivity of this Ab occurs with the MUC-1 expressed by infected cells. This was shown both by staining of infected cells as well as by western blot.

Parameters for the study of MVA-VLP vectors in mice have been previously established. Mice are to be administered a dose of 10⁷ TCID₅₀ MVA-VLP-MUC-1 by intramuscular (IM) administration in hind legs. The vaccine is typically formulated at a concentration of 10⁸ TCID₅₀/mL, so 100 μL is administered per animal.

Vaccine Immunogenicity Experiment

The immunogenicity of MVA-VLP-MUC-1 is assessed in a mouse experimental system. MUC-1 Tg mice are immunized with 10⁷ TCID₅₀ by IM injection using a prime-boost regimen. MVA-VLP-MUC-1 group 1 are compared with Tn-100-mer peptide loaded on BM-derived DC matured with Poly-ICLC (group 2). Groups 3 and 4 also receive soluble Tn-100-mer peptide to enhance antibody production and focus the response to the tandem repeat region. The test groups are as follows:

Immunogenicity Study Groups (10 mice per group)

| Group | Vaccination Condition |
|---|---|
| 1 | MVA-VLP-MUC-1 |
| 2 | Tn-100-mer on DC |
| 3 | Tn-100-mer on DC + soluble Tn-100-mer |
| 4 | MVA-VLP-MUC-1 + soluble Tn-100-mer |
| 5 | MVA-VLP-MUC-1 prime, Tn-100-mer plus Hiltonol boost |

Administration occurs on days 0 and boost on day 28 of the study. 10 days after the final vaccination, 3 mice from each group will be sacrificed, splenectomized, and their spleens used to measure T cell responses to vaccination. Serum is collected prior to administration, prior to the boost and 2 weeks after the boost and anti-MUC-1 IgG titers determined in ELISA on 100-mer and Tn-100-mer. 2 weeks after the boost, the remaining mice are challenged with MUC-1+ tumors SQ.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

All references cited herein are incorporated by reference in their entirety.

1. A immunogenic composition comprising:
a) a recombinant modified vaccinia ankara (MVA) viral vector comprising a sequence encoding hypoglycosylated MUC-1 or fragment thereof and a matrix protein sequence, and
b) a MUC-1 peptide.

2. The composition of claim 1 wherein the MUC-1 peptide comprises an immunogenic intracellular domain fragment of MUC-1 with sequence 407-475 of GenBank Protein Accession Number NP_001191214 or an immunogenic fragment thereof.

3. The composition of claim 1 wherein the MUC-1 peptide comprises an an immunogenic extracellular domain fragment of MUC-1 (for example sequence 20-376 of GenBank Protein Accession Number NP_001191214 or an immunogenic fragment thereof.

4. The composition of claim 1 wherein the MUC-1 peptide comprises a sequence TSAPDTRPAP (SEQ ID NO:1)

5. The composition of claim 1 wherein the MUC-1 peptide comprises a sequence AHGVTSAPDTRPAPGSTAPP (SEQ ID NO:2).

6. The composition of claim 1 wherein the MUC-1 peptide comprises a sequence AHGVTSAPDNRPALGSTAPP (SEQ ID NO:3).

7. The composition of claim 1 wherein the MUC-1 peptide comprises a sequence AHGVTSAPDTRPAPGSTAPPAHGVTSAPDNRPALGSTAPP (SEQ ID NO:4).

8. The composition of claim 1 wherein the MUC-1 peptide comprises a sequence
(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

9. The composition of claim 1 wherein the MUC-1 peptide comprises a sequence GenBank Protein Accession Number NP_001191214 (SEQ ID NO:6).

10. The composition of claim 1 wherein the MUC-1 peptide comprises a sequence SKKKKGCKLFAVWKITYKDTGTSAPDTRPAP (SEQ ID NO:7)) wherein the threonine at position 27 is optionally glycosylated with alpha-D-GalNAc.

11. The composition of claim 1, wherein at least one recombinant MVA vector expresses the MUC-1, peptide and a pharmaceutically acceptable carrier.

12. A method of inducing an immune response in a subject in need thereof comprising a) administering at least one recombinant MVA vector expressing MUC-1 to the subject in an amount sufficient to induce an immune response, and administering at least one MUC-1 peptide to boost the induced immune response.

13. The method of claim 12 wherein the MUC-1 peptide comprises an immunogenic intracellular domain fragment of MUC-1 with sequence 407-475 of GenBank Protein Accession Number NP_001191214 or an immunogenic fragment thereof.

14. The method of claim 12 wherein the MUC-1 peptide comprises an an immunogenic extracellular domain fragment of MUC-1 (for example sequence 20-376 of GenBank Protein Accession Number NP_001191214 or an immunogenic fragment thereof.

15. The method of claim 12 wherein the MUC-1 peptide comprises a sequence
(SEQ ID NO: 1)
TSAPDTRPAP

16. The method of claim 12 wherein the MUC-1 peptide comprises a sequence
(SEQ ID NO: 2)
AHGVTSAPDTRPAPGSTAPP.

17. The method of claim 12 wherein the MUC-1 peptide comprises a sequence
(SEQ ID NO: 3)
AHGVTSAPDNRPALGSTAPP.

18. The method of claim 12 wherein the MUC-1 peptide comprises a sequence
(SEQ ID NO: 4)
AHGVTSAPDTRPAPGSTAPPAHGVTSAPDNRPALGSTAPP.

19. The method of claim 12 wherein the MUC-1 peptide comprises a sequence
(Tn-100mer)
(SEQ ID NO: 5)
AHGVTSAPDTRPAPGSTAPPAHGVTSAPDTRPAPGSTAPPAHGVTSAPD
TRPAPGSTAPPAHGVTSAPDTRPAPGSTAPPAHGVTSAPDNRPALGSTA
PP.

20. The method of claim 12 wherein the MUC-1 peptide comprises a sequence GenBank Protein Accession Number NP_001191214 (SEQ ID NO:6).

21. The method of claim 12 wherein the MUC-1 peptide comprises a sequence SKKKKGCKLFAVWKITYKDTGTSAPDTRPAP (SEQ ID NO:7)) wherein the threonine at position 27 is optionally glycosylated with alpha-D-GalNAc.

22. The method of claim 12, wherein the immune response is a humoral immune response, a cellular immune response or a combination thereof.

23. The method of claim 12, wherein the immune response comprises: (i) production of binding antibodies or neutralizing antibodies against MUC-1, (ii) production of non-neutralizing antibodies against MUC-1; and/or (iii) production of a cell-mediated immune response against MUC-1.

24. (canceled)

25. (canceled)

26. A method of preventing or reducing the growth of a neoplasm comprising a) administering at least one recombinant MVA vector expressing MUC-1 to the subject in an amount sufficient to induce an immune response, and administering at least one MUC-1 peptide to boost the induced immune response.

27. A method of treating cancer comprising a) administering at least one recombinant MVA vector expressing MUC-1 to the subject in an amount sufficient to induce an immune response, and administering at least one MUC-1 peptide to boost the induced immune response.
